(19) United States
(12) Patent Application Publication
(10) Pub. No.: US 2025/0009698 A1
(43) Pub. Date: Jan. 9, 2025

(54) NERVONIC ACID-BASED COMPOSITIONS AND METHODS

(71) Applicant: REGENTS OF THE UNIVERSITY OF MINNESOTA, Minneapolis, MN (US)

(72) Inventors: Reena V. Kartha, Arden Hills, MN (US); James C. Cloyd, Minneapolis, MN (US)

(21) Appl. No.: 18/755,956

(22) Filed: Jun. 27, 2024

Related U.S. Application Data
(60) Provisional application No. 63/524,791, filed on Jul. 3, 2023.

(51) Int. Cl. A61K 31/201; A61K 47/40

(52) U.S. Cl. CPC A61K 31/201; A61K 47/40

(57) ABSTRACT

Pharmaceutical compositions including nervonic acid or a pharmaceutically acceptable salt thereof and a cyclodextrin. Methods of treating a subject having or at risk of having adrenoleukodystrophy and/or a neurological disease by administering a pharmaceutical composition of the present disclosure to the subject.

### CROSS-REFERENCE TO RELATED APPLICATION

This application claims the benefit of U.S. Provisional Patent Application No. 63/524,791, filed Jul. 3, 2023, which is incorporated herein by reference in its entirety.

### GOVERNMENT FUNDING

This invention was made with government support under the TR003941 awarded by the National Institutes of Health. The government has certain rights in the invention.

### SUMMARY

This disclosure describes, in one aspect a pharmaceutical composition including nervonic acid or a pharmaceutically acceptable salt thereof, and a cyclodextrin.

This disclosure describes, in one aspect a pharmaceutical composition including nervonic acid or a pharmaceutically acceptable salt thereof, and a cyclodextrin.

In one or more embodiments, the cyclodextrin includes a substituted or unsubstituted β-cyclodextrin, a substituted or unsubstituted α-cyclodextrin; a substituted or unsubstituted γ-cyclodextrin; or any combination thereof. In one or more embodiments, the cyclodextrin includes sulfobutylether-β-cyclodextrin.

In one or more embodiments, the pharmaceutical composition is a drug product.

In one or more embodiments, the pharmaceutical composition is in a liquid dosage form.

In one or more embodiments, the pharmaceutical composition is in a solid dosage form.

In another aspect, this disclosure describes a method of treating a subject having or at risk of having adrenoleukodystrophy, a neurological disease, or both. The method includes administering any one of the pharmaceutical compositions disclosed herein to the subject.

In one or more embodiments, the pharmaceutical composition is administered prior to the subject showing symptoms of adrenoleukodystrophy.

In one or more embodiments, the adrenoleukodystrophy is of the childhood cerebral adrenoleukodystrophy phenotype, adrenomyeloneuropathy phenotype, Addison disease phenotype, or any combination thereof.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Schematic of the adrenoleukodystrophy (ALD) disease pathophysiology.

FIG. 2. Schematic of the downstream effect of mutations in the ABCD1 gene leading to dysfunction of the ALD peroxisomal membrane transporter protein (ALDP).

FIG. 3. Schematic of the hypothesized role of nervonic acid in modulating ALD pathology.

FIG. 4. A plot showing the level of various fatty acids after normal cells, childhood cerebral ALD (cALD) cells, and adrenomyeloneuropathy (AMN) cells were treated with 0 micromolar (μM), 5 μM, 20 μM, or 50 μM erucic acid (EA).

FIG. 5. A plot showing the level of various fatty acids after normal cells, childhood cerebral ALD (cALD) cells, and adrenomyeloneuropathy (AMN) cells were treated with 0 μM, 5 μM, 20 μM, or 50 μM nervonic acid (NA).

FIG. 6. A plot showing the level of ATP in AMN cells treated with 0 μM, 5 μM, 20 μM, or 50 μM erucic acid or nervonic acid.

FIG. 7. A plot showing the level of ATP in cALD cells treated with 0 μM, 5 μM, 20 μM, or 50 μM erucic acid or nervonic acid.

FIG. 8. Schematic of a readout of a mitochondrial stress test (MST).

FIG. 9. Fluorescent images of human dermal AMN fibroblasts after treatment with 0 μM, 5 μM, 20 μM, or 50 μM nervonic acid. Green is CELLROX (Life Technologies, Corp., Carlsbad, CA), which detects reactive oxygen species (ROS) in live cells. Blue is DAPI which detects DNA content and nuclei in live cells. The experimental design for these results is illustrated in FIG. 31.

FIG. 10. Quantification of the CELLROX (Life Technologies, Corp., Carlsbad, CA) fluorescence from FIG. 9. The experimental design for these results is illustrated in FIG. 31.

FIG. 11. Fluorescent images of human dermal AMN fibroblasts after treatment with 0 μM, 5 μM, 20 μM, or 50 μM nervonic acid. Red is MITOSOX (Life Technologies, Corp., Carlsbad, CA), which detects mitochondrial superoxide in live cells. Blue is DAPI, which detects DNA content and nuclei in live cells. The experimental design for these results is illustrated in FIG. 31.

FIG. 12. Quantification of the MITOSOX (Life Technologies, Corp., Carlsbad, CA) fluorescence from FIG. 11. The experimental design for these results is illustrated in FIG. 31.

FIG. 13. Metabolic characterization of AMN fibroblasts from an MST as illustrated in FIG. 8. (A) Basal respiration in AMN fibroblasts treated with 0 μM, 5 μM, 20 μM, or 50 μM nervonic acid in an MST assay. (B) ATP production in AMN fibroblasts treated with 0 μM, 5 μM, 20 μM, or 50 μM nervonic acid in an MST assay. For details regarding the MST readout for FIGS. 13-19 and FIG. 32, please refer to FIG. 8.

FIG. 14. Metabolic characterization of AMN fibroblasts. (A) Proton leak in AMN fibroblasts treated with 0 μM, 5 μM, 20 μM, or 50 μM nervonic acid in an MST assay as illustrated in FIG. 8. (B) Maximal respiration in AMN fibroblasts treated with 0 μM, 5 μM, 20 μM, or 50 μM nervonic acid in an MST assay as illustrated in FIG. 8.

FIG. 15. Metabolic characterization of AMN fibroblasts. (A) Spare respiratory capacity in AMN fibroblasts treated with 0 μM, 5 μM, 20 μM, or 50 μM nervonic acid in an MST assay as illustrated in FIG. 8. (B) Non-mito respiration in AMN fibroblasts treated with 0 μM, 5 μM, 20 μM, or 50 μM nervonic acid in an MST assay as illustrated in FIG. 8.

FIG. 16. Metabolic characterization of AMN fibroblasts. (A) Coupling efficiency percent in AMN fibroblasts treated with 0 μM, 5 μM, 20 μM, or 50 μM nervonic acid in an MST assay as illustrated in FIG. 8. (B) Spare respiratory capacity percent in AMN fibroblasts treated with 0 μM, 5 μM, 20 μM, or 50 μM nervonic acid in an MST assay as illustrated in FIG. 8.

FIG. 17. Plot showing basal respiration after AMN cells were treated with 20 μM nervonic acid for twenty-four hours along with one or more mitochondrial metabolic pathway inhibitors (UK, BP, or ETO) measured using long-chain fatty acid oxidation stress test.

FIG. 18. Plot showing maximal respiration after AMN cells were treated with 20 μM nervonic acid for twenty-four hours along with one or more mitochondrial metabolic pathway inhibitors (UK, BP, or ETO) measured using long-chain fatty acid oxidation stress test.

FIG. 19. Plot showing acute response after AMN cells were treated with 20 μM nervonic acid for twenty-four hours along with one or more mitochondrial metabolic pathway inhibitors (UK, BP, or ETO) measured using long-chain fatty acid oxidation stress test.

FIG. 20. A mouse model experimental design for dosing ALD double knockout (−/−) mice with various amounts of nervonic acid or erucic acid through administration via food intake.

FIG. 21. A plot showing the level of C26:0-lysophsphatidylcholine (LPC) in the plasma of the mice treated according to FIG. 20.

FIG. 22. A plot showing the level of C26:0 in the plasma of the mice treated according to FIG. 20.

FIG. 23. A plot showing the concentration of C22:1 (erucic acid) in the plasma of the mice treated according to FIG. 20.

FIG. 24. A plot showing the concentration of C24:1 (nervonic acid) in the plasma of the mice treated according to FIG. 20.

FIG. 25. A plot showing the concentration of C26:1 in the plasma of the mice treated according to FIG. 20.

FIG. 26. A plot showing the level of C26:0 in the liver, kidney, brain, heart, and adrenal of the mice treated according to FIG. 20.

FIG. 27. A plot showing the level of C22:1 (erucic acid) in the liver, kidney, brain, heart, and adrenal of the mice treated according to FIG. 20.

FIG. 28. A plot showing the level of C24:1 (nervonic acid) in the liver, kidney, brain, heart, and adrenal of the mice treated according to FIG. 20.

FIG. 29. A schematic showing the proposed mechanisms of action of nervonic acid.

FIG. 30. A schematic showing the assembly of a drug with a cyclodextrin.

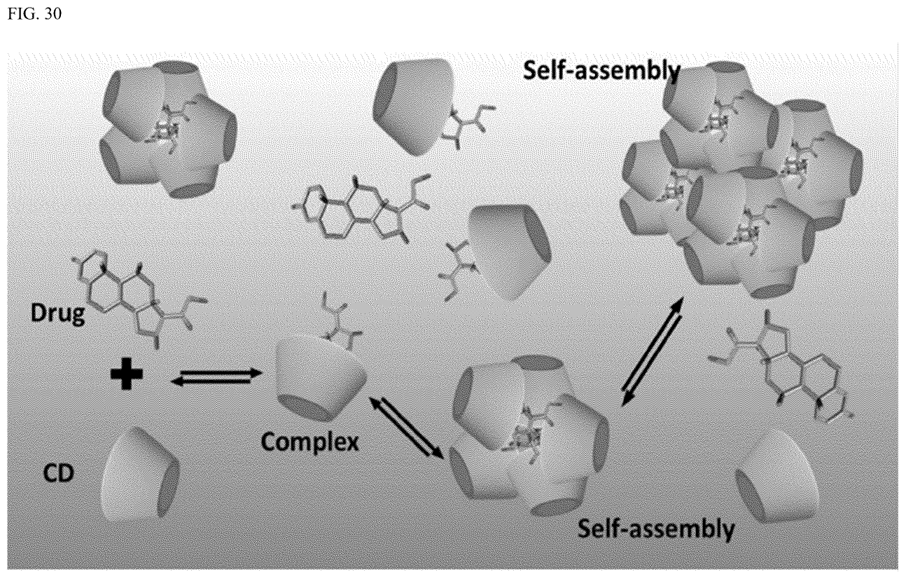

FIG. 31. An experimental design for an in vitro cell culture study on patient-derived adrenomyeloneuropathy (AMN) fibroblasts.

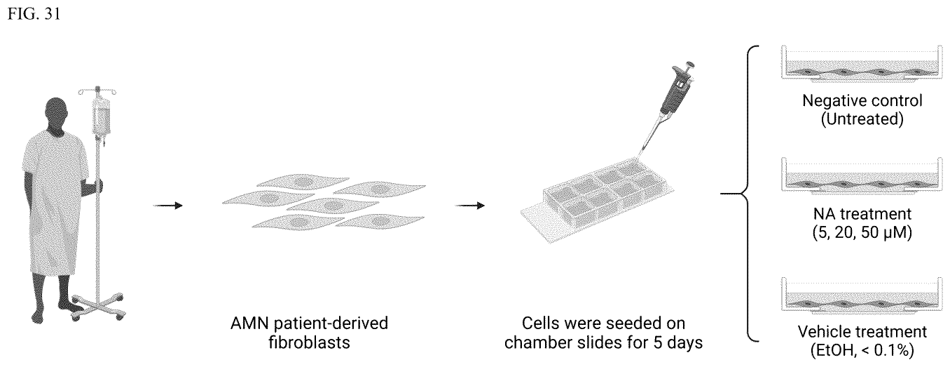

FIG. 32. Metabolic characterization of ALD cells in an MST assay as illustrated in FIG. 8. (A) Basal respiration in ALD cells and NHDF controls. (B) ATP production in ALD cells and NHDF controls. (C) Proton leak in ALD cells and NHDF controls. (D) Maximal respiration in ALD cells and NHDF controls. (E) Spare respiratory capacity in ALD cells and NHDF controls. (F) Non-mitochondrial respiration in ALD cells and NHDF controls. (G) Coupling efficiency percent in ALD cells and NHDF controls. (H) Spare respiratory capacity percent in ALD cells and NHDF controls. For all panels, ALD cells (left, blue); NHDF cells (right, red).

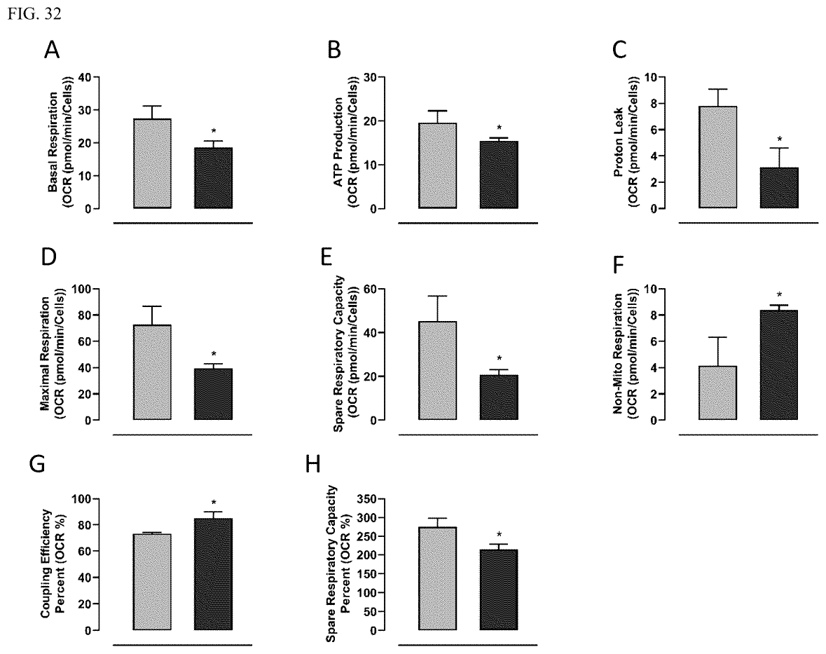

FIG. 33. A plot showing the level of C26:0-lysophsphatidylcholine (LPC) in the liver, kidney, brain, heart, and adrenal of the mice treated according to FIG. 20.

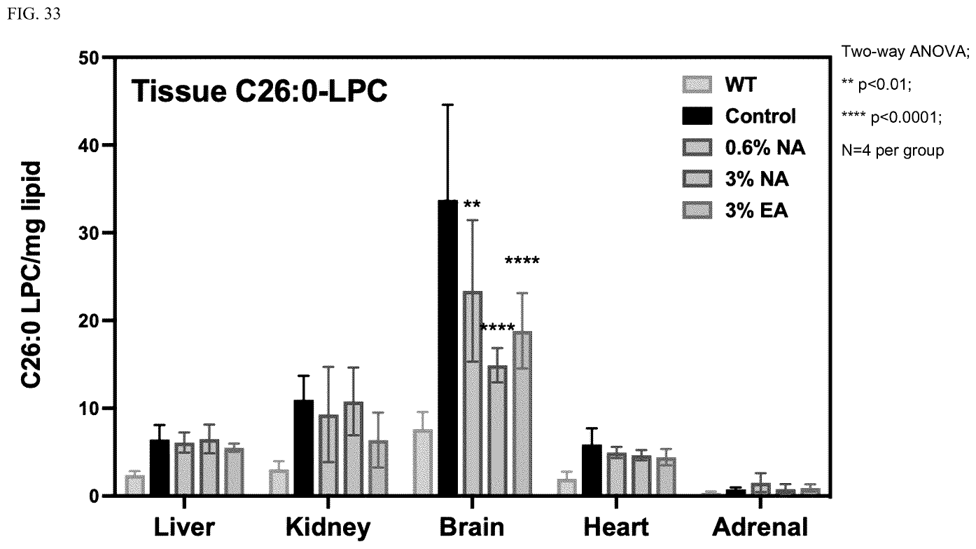

FIG. 34. A mouse model experimental design for dosing ALD gene-knockout mice (KO; ABCD1-) with a single dose of nervonic acid.

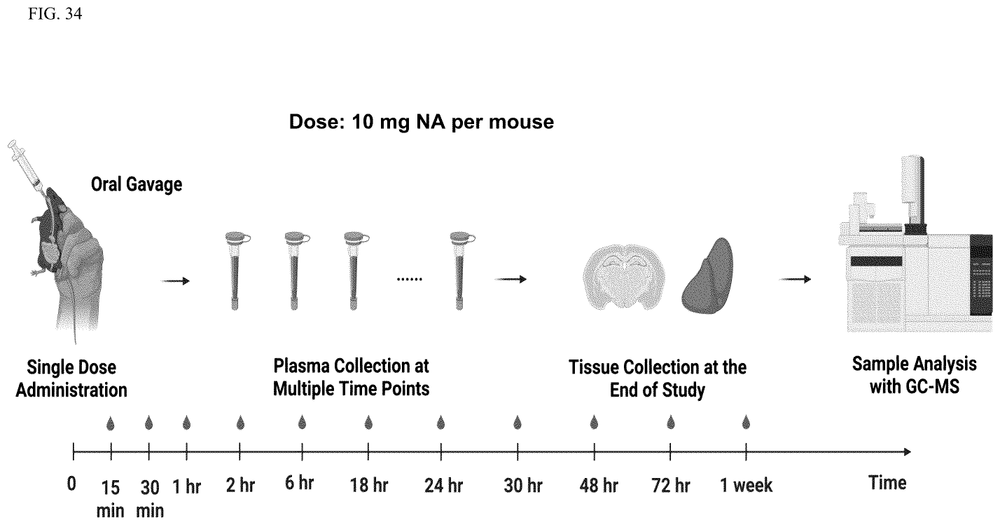

FIG. 35. Concentration-time profile of C24:1 (nervonic acid) in plasma of the mice treated according to FIG. 34. (A) 150-hour time scale. (B) 20-hour time scale showing detail of first six hours.

FIG. 36. Concentration-profile of C26:0 in plasma of the mice treated according to FIG. 34. (A) 150-hour time scale. (B) 20-hour time scale showing detail of first six hours.

FIG. 37. A plot showing C24:1 (nervonic acid) exposure in the brain of the mice treated according to FIG. 34.

FIG. 38. A plot showing C24:1 (nervonic acid) exposure in the liver of the mice treated according to FIG. 34.

FIG. 39. A plot showing changes in the level of C26:0-lysophsphatidylcholin (LPC) over time in the plasma of the mice treated according to FIG. 34.

FIG. 40. Phase-contrast images and fluorescent images of human dermal AMN fibroblasts after treatment with 0 μM, 5 μM, 20 μM, or 50 μM nervonic acid, and stained with MITOTRACKER (Molecular Probes, Inc., Sunnyvale, CA), which detects mitochondrial activity, and DAPI, which detects DNA content and nuclei in live cells. The experimental design for these results is illustrated in FIG. 31.

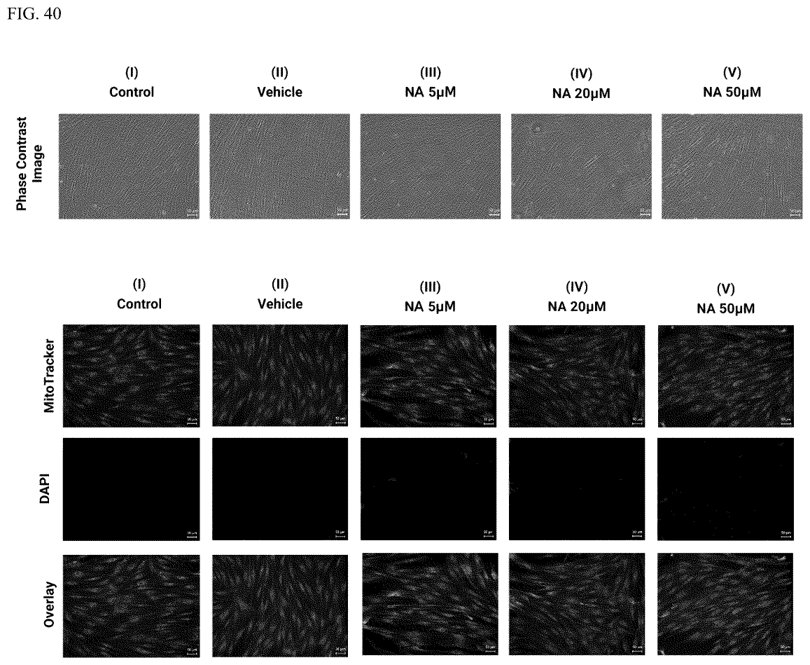

FIG. 41. Quantification of the MITOTRACKER (Molecular Probes, Inc., Sunnyvale, CA) fluorescence from FIG. 40. The experimental design for these results is illustrated in FIG. 31.

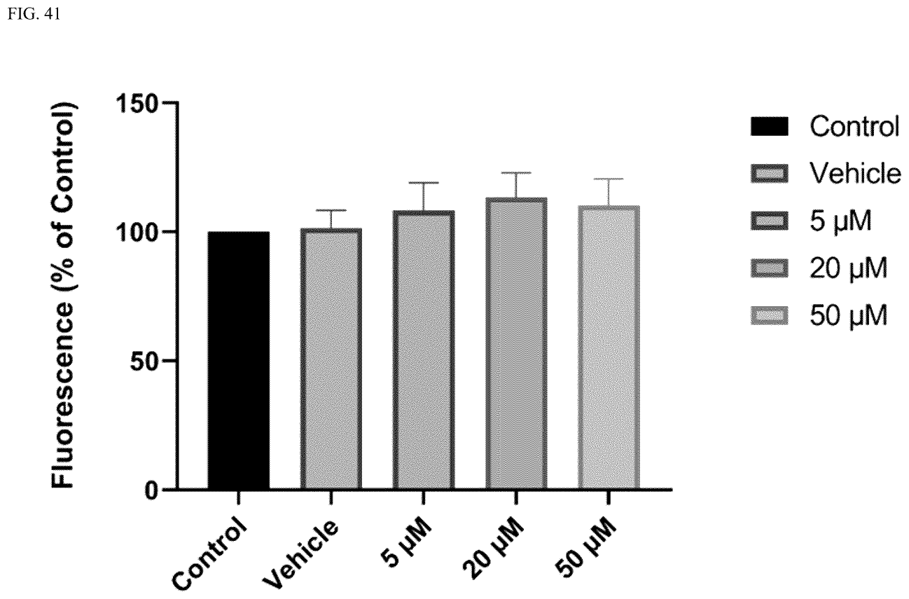

FIG. 42. Mitochondrial respiration in ALD cells and NHDF controls in an MST assay as illustrated in FIG. 8. ALD cells (circles); NHDF cells (squares).

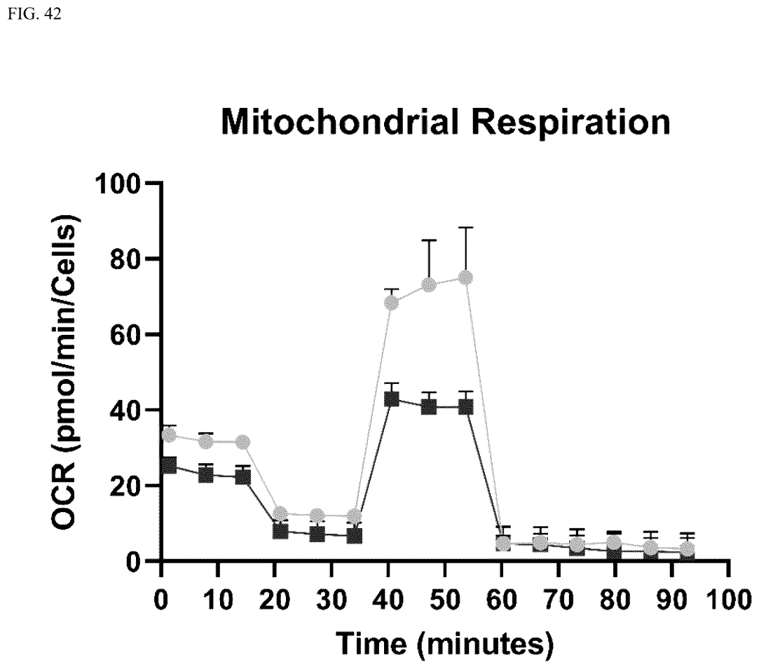

FIG. 43. Mitochondrial respiration in AMN fibroblasts treated with 0 μM, 5 μM, 20 μM, or 50 μM nervonic acid in an MST assay as illustrated in FIG. 8. Vehicle (black squares); Control (white circles); 5 μM (green triangles); 20 μM (red triangles); 50 μM (grey diamonds).

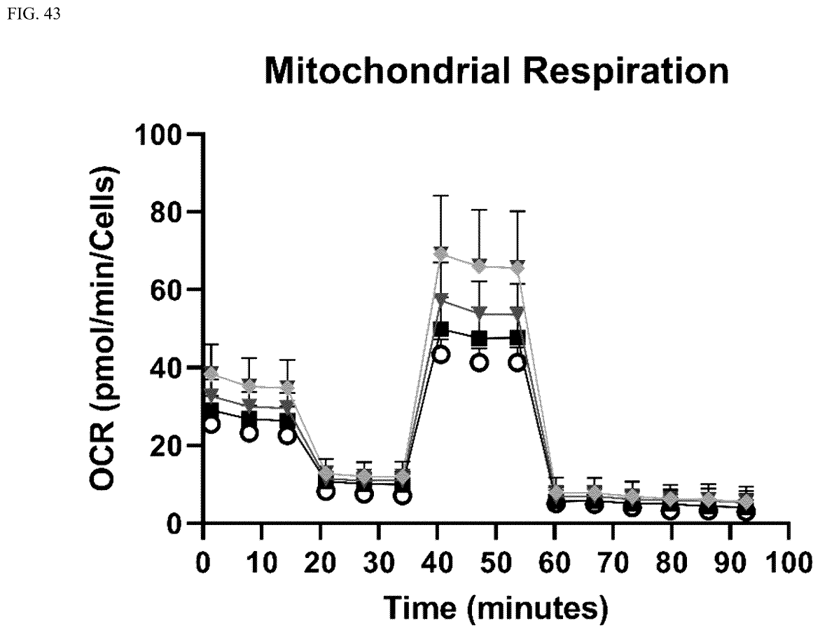

### DETAILED DESCRIPTION OF ILLUSTRATIVE EMBODIMENTS

The present disclosure describes pharmaceutical compositions that include nervonic acid and methods of administering such compositions. The pharmaceutical compositions include nervonic acid and a cyclodextrin. In one or more embodiments, the pharmaceutical compositions may be administered to a patient that has or is at risking of having an adrenoleukodystrophy disease (ALD) and/or a neurological disease.

Adrenoleukodystrophy (ALD) is a rare genetic condition that presents at a variety of different ages and has a range of phenotypes. ALD is more common in males than females and females typically present later in life as compared to males. Clinical manifestations of ALD vary significantly according to the disease severity and the age at presentation. ALD can be classified into three broad phenotypes based on the age of presentation and the organs affected. The first phenotype of ALD is Addison's disease which is characterized by adrenal gland dysfunction, and up to 50% patients show this phenotype in childhood. The manifestations associated with this Addison's disease ALD subtype are the result of decreased production of aldosterone resulting in hypotension and dehydration; cortisol resulting in generalized weakness; and hyperpigmentation of the skin. The second phenotype of ALD is cerebral ALD which accounts for 33%-40% of the ALD patient population and typically affects children between the age of three to ten years. The hallmark feature of cerebral ALD is developmental regression such as progressive sensory and severe neurological deficits, severe disability, coma, and death are generally followed by more progressive sensory and severe neurological deficits. Childhood cerebral ALD (cALD) is tested for in newborn screening often by analyzing the level of the C26:0-lysophsphatidylcholine (LPC) fatty acid. A small percentage of adults may present a similar pattern to childhood cerebral ALD. The third phenotype of ALD is adrenomyeloneuropathy (AMN) which typically presents in the third decade of life as a milder form of ALD. AMN is characterized by walking difficulties, unbalanced gait, and bowel/bladder sphincter dysfunction. Additional phenotypes of ALD include olivopontocerebellar and asymptomatic.

Adrenoleukodystrophy (ALD) is an X-linked rare genetic condition caused by mutations in the ABCD1 gene, which encodes a peroxisomal ATP-binding cassette transporter protein (ALDP). ALDP is important for very long chain fatty acid (VLCFA) homeostasis. VLCFAs, such as C26:0, are transported into peroxisomes for degradation via the ALDP. Inhibition of VLCFA entry into the peroxisomes results in high levels of VLCFAs which can trigger oxidative stress and other downstream effects.

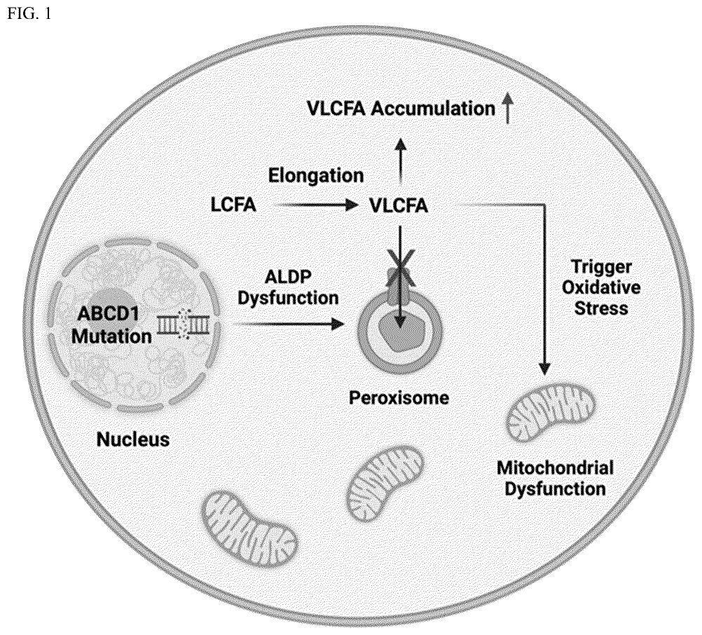

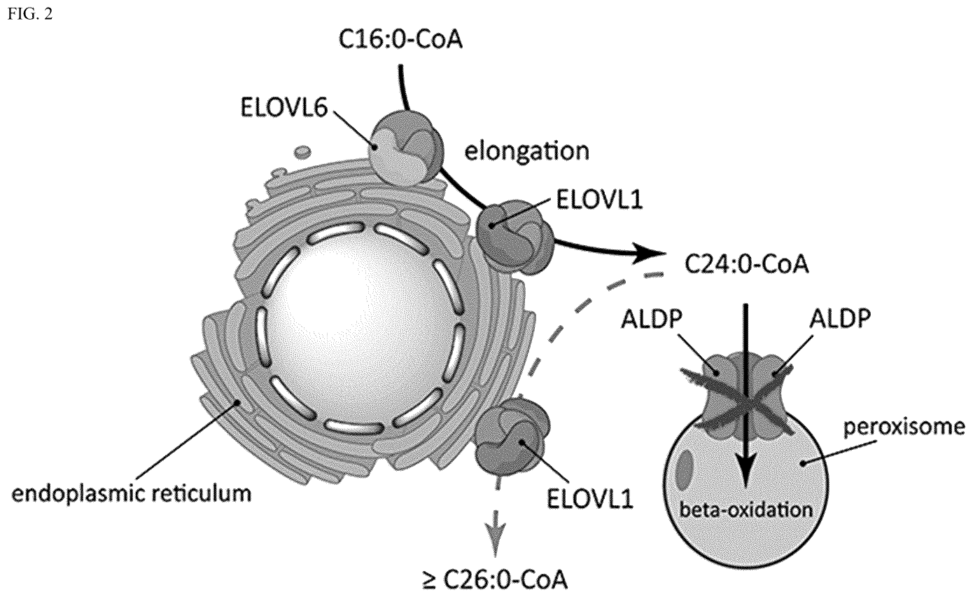

FIG. 1 and FIG. 2 show schematics of ALD pathophysiology and biochemistry. Saturated and monounsaturated VLCFA are synthesized from long-chain fatty acids (LFA) via the concerted action of elongate enzymes ELOVL6 (C18:0-C22:0) and ELOVL1 (C24:0-C26:0). ALDP transports VLCFacyl-coenzyme A (CoA) across the peroxisomal membrane for degradation. A deficiency in ALDP impairs peroxisomal β-oxidation of VLCFA but also raises cytosolic levels of VLCFacyl-CoA which are substrate for further elongation by ELOVL1. Mutations in the ABCD1 gene result in a defective transporter, leading to accumulations of saturated very long chain fatty acids (VLCFA), predominantly hexacosanoic acid (also called cerotic acid or C26:0), particularly in plasma, brain white matter, spinal cord, and adrenal cortex. Accumulated VLCFAs would then be incorporated into complex lipids, resulting in tissue injuries such as destabilization of the myelin sheath (important for normal brain function), and the onset of demyelinating pathology.

There is no cure for ALD; however, there are several treatment options. Allogeneic hematopoietic stem cell transplantation (HSCT) is an approved therapy known to stabilize the disease progression of cALD. Previous reports document favorable outcomes when HSCT is performed early in the course of the disease, with better survival post-transplantation when performed in presymptomatic boys. However, HSCT is associated with serious and sometimes fatal complications, including infection and graft failure or rejection. The timely availability of a suitably histocompatible related or unrelated donor also remains a significant limitation of allogeneic HSCT.

Lorenzo's oil (LO) was once considered a therapeutic option for ALD, especially in asymptomatic or pure AMN patients. LO is a mixture of erucic acid (C22:1) and oleic acid (C18:1) that has been shown to significantly reduce plasma C26:0 levels and has the potential to slow down the development of cALD during childhood. However, it did not show a significant effect on preventing the development of demyelinating lesions in ALD. Moreover, preliminary animal studies showed erucic acid to be associated with cardiotoxicity, thus making it a controversial therapeutic choice for ALD. Due to contradictory and unclear benefits, the commercial development of LO was terminated recently in the USA.

Nervonic acid (Chemical Abstract Services registry number (CAS No.): 506-37-6), also called NA, C24:1 cis, or just C24:1, is a monounsaturated fatty acid having a C24 chain with a cis double bond between the 9^{th} and 10^{th} carbons in the chain (numbering starting opposite the carboxylic acid). NA is a component of brain white matter and the myelin sheath of the nervous system. As such, NA is associated with brain development and maintenance as well as the biosynthesis and improvement of nerve cells.

In ALD patients, the levels of NA are affected. For example, elevated C26:0 was accompanied by decreased NA biosynthesis in skin fibroblasts isolated from ALD patients. Additionally, decreased levels of NA were observed in post-mortem ALD brains. Studies conducted using NA have shown that similar to Lorenzo's oil (mainly EA), NA can competitively inhibit ELOVL1 and reduce C26:0 accumulation. Furthermore, it has been shown that NA can biochemically reverse the accumulation of C26:0 in ALD patient-derived fibroblasts.

Due at least in part to the limited water solubility, formulating NA is challenging. The estimated Log P of NA is 10.890, suggesting NA has a higher affinity for the lipid/organic solvent phase than the water phase. The solubility of nervonic acid in ethanol is approximately 10 mg/ml and approximately 20 mg/ml in dimethyl sulfoxide and dimethylformamide. However, without an excipient, the estimated water solubility of NA is 9.3×10⁻⁹ mg/ml.

In one aspect, the present disclosure describes pharmaceutical compositions that include nervonic acid (NA) and a cyclodextrin excipient. NA may be referred to as a drug substance. In one or more embodiments, the pharmaceutical compositions are drug products.

As used herein, the term “drug substance” refers to an active ingredient that is intended to furnish pharmacological activity or other direct effect in the diagnosis, cure, mitigation, treatment, or prevention of disease or to affect the structure or any function of a body of a subject (such as the human body).

As used herein, the term “drug product” refers to the finished dosage form, such as a tablet, capsule, or solution, that contains a drug substance, generally, but not necessarily, in association with one or more other ingredients.

As used herein, a “finished dosage form” means (A) a drug product in the form in which it will be administered to a patient, such as a tablet, capsule, or solution; (B) a drug product in a form in which reconstitution is necessary prior to administration to a subject (such as a patient), such as oral suspensions or lyophilized powders; (C) a drug product in a form that may be manipulated prior to administration to achieve a desired dose of the drug substance; or (D) any combination of an active pharmaceutical ingredient with another component of a drug product for purposes of production of a drug product described in (A), (B), or (C). A finished dosage form may be a drug product in a concentrated form that is to be diluted prior to administration.

In one or more embodiments, the pharmaceutical composition (e.g., drug product) is in solid unit dosage form. Solid unit dosage forms are administered via enteral administration. In addition to the drug substance (i.e., nervonic acid) and the cyclodextrin excipient, solid dosage forms may include one or more additional excipients. Excipients are substances other than the drug substance included in formulations. Excipients may function to bulk up formulations; stabilize formulations; facilitate formulation; enhance the therapeutic effect of the drug substance, for example, by facilitating absorption or enhancing solubility; mask the taste of the dosage form; or any combination thereof. Examples of excipients that may be included in a solid dosage form include fillers, lubricants, glidants, binders, disintegrants, disaggregates, sweeteners, colors, preservatives, and sorbents. Solid unit dosage forms may include a coating to impart properties to the dosage forms. For example, solid unit dosage forms may include a sweetener coating to mask the taste, a polymer to impart smoothness, an ingredient to control the release rate of the drug substance, or the like.

Solid unit dosage forms include tablets, capsules, and powders. Tablets are compressed or molded solids. For example, tablets may be formed by bringing together the drug substance with one or more excipients or other agents and molding or pressing the ingredients into the desired geometry. Capsules are hard-shelled or soft-shelled envelopes encapsulating a liquid form of the drug substance (e.g., the drug substance is a liquid, or the drug substance is dissolved in a carrier) or a solid form of the drug substance (e.g., powder or miniature pellets). Hard-shell capsules are often formed by adding the drug substance to a first body having a first diameter and sealing the first body with a second body having a second diameter that is larger than the first diameter of the first body. Unlike hard-shell capsules, soft-shelled capsules have a single body that is at least partially filled with the drug substance (e.g., via blow molding) and sealed with a drop of sealant (e.g., gelatin).

Solid unit dosage forms may be formulated as slow or sustained release solid unit dosage forms. Sustained release solid dosage forms are formulated such that the drug substance is released over a period of time and/or released in a desired location of the digestive tract. Examples of sustained release formulations include drug substances embedded in an insoluble porous matrix or a swellable gel matrix. Another sustained release method is the osmotic controlled release oral delivery system. In this system, a drug substance is enclosed in a water-permeable membrane having a laser drilled hole. When water passes through the membrane, the drug substance is pushed out the hole.

In one or more embodiments, the pharmaceutical composition (e.g., drug product) is in a liquid dosage form. A liquid dosage form may be a solution or a suspension that can be delivered parenterally (e.g., intravenously, subcutaneously, intramuscularly, intra-particularly) or orally. The liquid form includes a pharmaceutically acceptable carrier. As used herein, the term “carrier” includes any solvent, dispersion medium, vehicle, diluent, carrier solution, suspension, colloid, and the like. The use of such media and/or agents for drug substances is well known in the art. Except insofar as any conventional media or agent is incompatible with the drug substance, its use in the pharmaceutical composition (e.g., drug products) of the present disclosure is contemplated.

In one or more embodiments where the pharmaceutical composition (e.g., drug product) is in a liquid dosage form, the pharmaceutical composition includes a carrier. In one or more embodiments, the carrier is or includes water. In one or more embodiments, the water carrier is a buffered aqueous solution, that is, the solution includes one or more agents that allow the solution to resist change in pH when exposed to certain amounts of acid and/or base. A buffered aqueous solution includes at least one weak acid and its conjugate base or at least one weak base and its conjugate acid. The weak acid and its conjugate base or the weak base and its conjugate acid are derived from buffering agents that are weak acids, weak bases, or salts thereof. As such, in one or more embodiments, the drug product comprises one or more buffering agents and the ions thereof. Buffering agents are salts of weak acids and/or salts of weak bases. Examples of buffering agents include, but are not limited to, acetic acid or salts thereof (e.g., acetate salts); monosodium and/or disodium phosphate or other phosphate salts; maleic acid or salts thereof (e.g., maleate salts); citric acid or salts thereof (e.g., citrate salts); histidine or salts thereof (e.g., histidine sodium salt); tartaric acid or salts thereof (e.g., tartrate salts); lactic acid or salts thereof (e.g., lactate salts); succinic acid or salts thereof (e.g., succinate salts); gluconic acid or salts thereof (e.g., gluconate salts); fumaric acid or salts thereof (e.g., fumarate salts); carbonic acid or salts thereof (e.g., bicarbonate salts), tromethamine and salts thereof; and phthalic acid and salts thereof (e.g., hydrogen phthalate salts).

In one or more embodiments, the carrier is water and the water solution is not buffered. That is, the water solution does not include buffering agents.

In one or more embodiments, the carrier includes water and one or more additional solvents. Examples of additional solvents include ethanol and dimethyl sulfoxide (DMSO). In one or more embodiments where one or more additional solvent are present in the carrier the total volume-percent (vol-%) of the one or more additional solvents is 1 vol-% or greater, 5 vol-% or greater, 10 vol % or greater, 15 vol-% or greater, 20 vol-% or greater, 25 vol-% or greater, 30 vol-% or greater, or 35 vol-% or greater. In one or more embodiments where one or more additional solvent are present in the carrier the total volume-percent (vol-%) of the one or more additional solvents is 1 vol-% or greater, 40 vol-% or less, 35 vol-% or less, 30 vol-% or less, 25 vol-% or less, 20 vol-% or less, 15 vol-% or less, 10 vol-% or less, or 5 vol-% or less.

In one or more embodiments, the carrier may include various salts such as, for example, sodium chloride, potassium chloride, calcium chloride, magnesium chloride, and sodium hydroxide. In one or more embodiments, the drug product includes one or more isotonic agents, such as sodium chloride and/or dextrose.

In addition to nervonic acid and the cyclodextrin excipient, the liquid dosage form may include one or more additional excipients. Examples of excipients used in liquid dosage forms include, buffering agents, salts, sweeteners, preservatives, amongst others.

The pharmaceutical composition (e.g., drug product) may have a certain pH. The pH may enhance the solubility of the drug substance. The pH of a solution can be measured or calculated using techniques known in the art, for example, according to US Pharmacopeia 791. In one or more embodiments, the pharmaceutical composition has a pH of 4.0 or greater, 5.0 or greater, 6.0 or greater, 6.5 or greater, 7.0 or greater, 7.5 or greater, 8.0 or greater, or 8.5 or greater. In one or more embodiments, the pharmaceutical composition has a pH of 9.0 or less, 8.0 or less, 7.5 or less, 7.0 or less, 6.5 or less, 6.0 or less, or 5.5 or less. In one or more embodiments, the pharmaceutical composition has a pH of 4.0 to 9.0, 4.0 to 8.0, 4.0 to 7.5, 4.0 to 7.0, 4.0 to 6.5, 4.0 to 6.0, or 4.0 to 5.0. In one or more embodiments, the pharmaceutical composition has a pH of 5.0 to 9.0, 5.0 to 8.0, 5.0 to 7.5, 5.0 to 7.0, 5.0 to 6.5, or 5.0 to 6.0. In one or more embodiments, the pharmaceutical composition has a pH of 6.0 to 9.0, 6.0 to 8.0, 6.0 to 7.5, 6.0 to 7.0, or 6.0 to 6.5. In one or more embodiments, the pharmaceutical composition has a pH of 6.5 to 9.0, 6.5 to 8.0, 6.5 to 7.5, or 6.5 to 7.0. In one or more embodiments, the pharmaceutical composition has a pH of 7.0 to 9.0, 7.0 to 8.0, or 7.0 to 7.5. In one or more embodiments, the drug product has a pH of 7.5 to 9.0 or 7.5 to 8.0. In one or more embodiments the pharmaceutical composition has a pH of 8.0 to 9.0.

Various acids and/or bases may be used to adjust the pH of a pharmaceutical formulation to produce the pharmaceutical composition. In the case that the pH of the pharmaceutical composition is adjusted using an acid and/or base or salt thereof, the pharmaceutical composition includes the ions of the acid and/or base or salt thereof used to adjust the pH. Examples of acids and/or bases that may be used to adjust the pH of the pharmaceutical composition include, but are not limited to, strong acids and/or strong bases or salts thereof, such as sodium hydroxide, potassium hydroxide, lithium hydroxide, strontium hydroxide, barium hydroxide, calcium hydroxide, cesium hydroxide, hydrochloric acid, sulfuric acid, nitric acid, phosphoric acid, perchloric acid, chloric acid, hydrobromic acid, hydroiodic acid; or weak acids and/or weak basis or salts thereof such as those described relative to the buffering agents herein. In one or more embodiments where the pH of the pharmaceutical composition is adjusted, sodium hydroxide and/or hydrochloric acid is used to adjust the pH.

The pharmaceutical composition may have a certain osmolality. Osmolality is the total concentration of solute particles per kilogram. The osmolality can be measured by freezing point depression measurement or using an osmometer such as described in US Pharmacopeia 785. The osmolality of the pharmaceutical composition may be adjusted through the addition of sodium chloride, one or more buffering agents, and/or dextrose to produce the drug product. In such embodiments where the osmolality of the pharmaceutical composition is adjusted, the compound and/or the ions of the compound used to adjust the pharmaceutical composition are included in the pharmaceutical composition.

In the pharmaceutical compositions of the present disclosure, the drug substance (i.e., nervonic acid) may be provided as a free acid or in the form of a pharmaceutically acceptable hydrate, solvate, or salt. In the context of drug products that includes a liquid carrier, the free acid or pharmaceutically acceptable hydrate, solvate, or salt forms of a drug substance may be ionized and as such, the pharmaceutical composition (e.g., drug product) may include an ion that was previously bound to the drug substance via an ionic bond. In the present disclosure, the drug substance is described as its form before formulation into the pharmaceutical composition.

The term “free acid” refers to the acid (protonated) form of a carboxylic acid. As used herein, a nervonic acid that is describes as a free acid includes a carboxylic acid that is in the free acid form.

A pharmaceutically acceptable salt of a drug substance refers to an ionized or ionizable drug substance that has been neutralized through one or more ionic bonds to an appropriate counterion (e.g., and anion or cation). For example, a nervonic acid can be made into a salt through reaction with a base. In instances where nervonic acid is in the form of a pharmaceutically acceptable salt, the salt may be classified in view of the counterion that is used to neutralize the negative charge of the deprotonated carboxylic acid. For example, the pharmaceutically acceptable salt of a nervonic acid may be denoted as the counterion cation name followed by nervonate. Examples of pharmaceutically acceptable salts of nervonic acid include sodium nervonate and potassium nervonate.

In the present disclosure and in the claims, any reference to a “nervonic acid” refers broadly to nervonic acid in the form of a free acid, salts of nervonic acid (nervonate salts), anhydrous nervonic acid, anhydrous nervonate salts, hydrates or solvates of nervonic acid, and hydrates or solvates of nervonate salts as suitable alternatives, unless otherwise specified. In one or more embodiments, the drug substance is, or includes, a free acid form of nervonic acid. In one or more embodiments, the drug substance is, or includes, a pharmaceutically acceptable salt of nervonic acid (i.e., a nervonate salt).

The amount of nervonic acid in the pharmaceutical compositions is described as nervonic acid equivalents. As used herein, the term “nervonic acid equivalents” refers to the amount of nervonic acid and nervonate present in the dilutable aqueous pharmaceutical composition, which results from inclusion of nervonic acid and/or a salt thereof. When nervonic acid is the only source of nervonic acid or nervonate in the pharmaceutical composition, the nervonic acid equivalents is the amount of nervonic acid included in the pharmaceutical composition. When a nervonate salt is included, the nervonic acid equivalents is the concentration of the nervonate salt expressed as the equivalent amount of nervonic acid. For example, a pharmaceutical composition that includes of 100 mg of sodium nervonate is the same as a pharmaceutical composition that includes 9.4 mg nervonic acid equivalents (0.1 g×(sodium nervonate mol/389.61 g)=0.00026 mol sodium nervonate; 0.00026 mol×366.62 g/mol nervonic acid=0.094 g nervonic acid).

The amount of nervonic acid equivalents in the pharmaceutical composition administered to a subject can vary depending on various factors including, but not limited to, the weight, physical condition, and/or age of the subject, and/or the route of administration. As such, the absolute amount of nervonic acid equivalent administered to a subject may be different than the absolute amount of nervonic acid in the pharmaceutical composition (e.g., drug product) as described herein. For example, solid unit dosage forms of the pharmaceutical composition may be split in order to decrease the total amount of nervonic acid equivalents administered in a single dose. Likewise, more than one solid dosage unit form may be administered in a single dose to increase the total amount of nervonic acid equivalents delivered to the subject.

In one or more embodiments, the drug product is a solid such as a tablet or capsule. In one or more such embodiments, the drug product includes 1 mg or greater 1.5 mg or greater, 2 mg or greater, 2.5 mg or greater, 3 mg or greater, 3.5 mg or greater, 4 mg or greater, 4.5 mg or greater, 5 mg or greater, 5.5 mg or greater, 6 mg or greater, 6.5 mg or greater, 7 mg or greater, 7.5 mg or greater, 8 mg or greater, 8.5 mg or greater, 9 mg or greater, 9.5 mg or greater, 10 mg or greater, 12.5 mg or greater, 15 mg or grater, 17.5 mg or greater, 20 mg or greater, 25 mg or greater, 30 mg or greater, or 40 mg or greater, 50 mg or greater, 75 mg or greater, 100 mg or greater, 125 mg or greater, 150 mg or greater, 175 mg or greater, 200 mg or greater, or 250 mg or greater nervonic acid equivalents. In one or more embodiments, the drug product includes 300 mg or less, 250 mg or less, 200 mg or less, 175 mg or less, 150 mg or less, 125 mg or less, 100 mg or less, 75 mg or less, 50 mg or less, 40 mg or less, 30 mg or less, 25 mg or less, 20 mg or less, 17.5 mg or less, 15 mg or less, 12.5 mg or less, 10 mg or less, 9.5 mg or less, 9 mg or less, 8.5 mg or less, 8 mg or less, 7.5 mg or less, 7 mg of or less, 6.5 mg or less, 6 mg or less, 5.5 mg or less, 5 mg or less, 4.5 mg or less, 4 mg or less, 3.5 mg or less, 3 mg or less, 2.5 mg or less, 2 mg or less, or 1.5 mg or less nervonic acid equivalents.

In one or more embodiments, the pharmaceutical composition (e.g., drug product) is in a liquid dosage form. In one or more such embodiments, the drug product includes 0.5 mg/ml or greater, 1 mg/ml or greater 1.5 mg/ml or greater, 2 mg/ml or greater, 2.5 mg/ml or greater, 3 mg/ml or greater, 3.5 mg/ml or greater, 4 mg/ml or greater, 4.5 mg/ml or greater, 5 mg/ml or greater, 5.5 mg/ml or greater, 6 mg/ml or greater, 6.5 mg/ml or greater, 7 mg/ml or greater, 7.5 mg/ml or greater, 8 mg/ml or greater, 8.5 mg/ml or greater, 9 mg/ml or greater, 9.5 mg/ml or greater, 10 mg/ml or greater, 12.5 mg/ml or greater, 15 mg/ml or grater, 17.5 mg/ml or greater, 20 mg/ml or greater, 25 mg/ml or greater, 30 mg/ml or greater, or 40 mg/ml or greater, 50 mg/ml or greater, 75 mg/ml or greater, 100 mg/ml or greater, 125 mg/ml or greater, 150 mg/ml or greater, 175 mg/ml or greater, 200 mg/ml or greater, or 250 mg/ml or greater nervonic acid equivalents. In one or more embodiments, the drug product includes 300 mg/ml or less, 250 mg/ml or less, 200 mg/ml or less, 175 mg/ml or less, 150 mg/ml or less, 125 mg/ml or less, 100 mg/ml or less, 75 mg/ml or less, 50 mg/ml or less, 40 mg/ml or less, 30 mg/ml or less, 25 mg/ml or less, 20 mg/ml or less, 17.5 mg/ml or less, 15 mg/ml or less, 12.5 mg/ml or less, 10 mg/ml or less, 9.5 mg/ml or less, 9 mg/ml or less, 8.5 mg/ml or less, 8 mg/ml or less, 7.5 mg/ml or less, 7 mg/ml of or less, 6.5 mg/ml or less, 6 mg/ml or less, 5.5 mg/ml or less, 5 mg/ml or less, 4.5 mg/ml or less, 4 mg/ml or less, 3.5 mg/ml or less, 3 mg/ml or less, 2.5 mg/ml or less, 2 mg/ml or less, 1.5 mg/ml or less or less nervonic acid equivalents. In such pharmaceutical compositions (e.g., drug product), the absolute amount of nervonic acid equivalents delivered to a subject is dependent on the volume of the pharmaceutical composition delivered to the subject.

The pharmaceutical compositions of the present disclosure include a cyclodextrin. As used herein, the term “cyclodextrin” refers to a glucose oligosaccharide where the glucose subunits are covalently joined via an α-1,4-glycosic bonds to form a macrocycle. A cyclodextrin is an excipient than can increase the water solubility of a drug substance, such as nervonic acid. More specifically, cyclodextrins are cup or coned shaped compounds having a hydrophobic interior and a hydrophilic exterior. The hydrophobic interior allows cyclodextrins to noncovalently complex with hydrophobic drugs while the hydrophilic exterior allows the cyclodextrin-drug complex to be more water soluble than the drug alone.

Cyclodextrins are described by the number of glucose subunits. The number of glucose subunits in a cyclodextrin can vary. In one or more embodiments, the pharmaceutical compositions of the present disclosure include a cyclodextrin that has six (α-cyclodextrin; α-CD), seven (β-cyclodextrin; β-CD), eight (γ-cyclodextrin), nine, or ten glucose units.

Cyclodextrins may be unsubstituted or substituted. An unsubstituted cyclodextrin is a cyclodextrin where none of the hydroxyl groups of the glucose subunits are replaced with a different moiety. A substituted cyclodextrin is a cyclodextrin where one or more of the hydroxyl groups of the glucose subunits of the cyclodextrin are substituted with a different moiety. The substituent may be described as the entire group that replaces the hydroxyl or as an R group where the location of the substituent can be described as —OR or —OCH₂R where R is a moiety other than H. Example substitutions include 2-hydroxypropyl (R=—CH₂CH(OH)(CH₃)), methoxy (R=—OCH₃), and sulfobutylether (R=CH₂CH₂CH₂CH₂(SO₃)⁻). In cases where the substituent has a formal charge, a counter ion may be present. For example, when the substituent is sulfobutylether, the substituent may also include a counter cation such as sodium.

The pattern of cyclodextrin substitution may vary. A single hydroxyl on the entire cyclodextrin may be substituted or two or more hydroxyls on the cyclodextrin may be substituted. In some cases where two or more hydroxyls are substituted, the two or more hydroxyls may be on the same glucose subunit, different glucose subunits, or both. When a plurality of substituted cyclodextrin compounds from the same class are used in a pharmaceutical composition, the number and pattern of the substitution on each cyclodextrin in the plurality may be the same or different.

The average number of substitutions on a cyclodextrin in a plurality of cyclodextrins having the same substituent is described as the average degree of substitution. In one or more embodiments, the average degree of substitution is 1 or greater, 2 or greater, 3 or greater, 4 or greater, 5 or greater, 6 or greater, 7 or greater, 8 or greater, 9 or greater, 10 or greater, 11 or greater, 12 or greater, 13 or greater, 14 or greater, 15 or greater, 16 or greater, 17 or greater, 18 or greater, or 19 or greater. In one or more embodiments, the average degree of substitution is 20 or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 3 or less, or 2 or less. In one or more embodiments, the average degree of substitution is 6 to 8.

Substituted cyclodextrins can be described by the identity of the substituent and the number of glucose subunits denoted as X-Y-cyclodextrin where X is the identity of the substituent and Y is the number of glucose subunits. For example, a hydroxypropyl substituted cyclodextrin having seven glucose subunits can be describes as hydroxypropyl-β-cyclodextrin. Additionally, the average degree of substitution may be denoted for substituted cyclodextrins using the formula (X)ₙ-Y-cyclodextrin where X is the identity of the substituent, n is the average degree of substitution, and Y is the number of glucose subunits.

Unsubstituted cyclodextrins can be denoted as unsubstituted Y-cyclodextrin where Y is the number of the glucose subunits. For example, unsubstituted β-cyclodextrin refers to a cyclodextrin having 7 glucose subunits and no substitutions.

In one or more embodiments, the pharmaceutical compositions include unsubstituted α-cyclodextrin. In one or more embodiments, the pharmaceutical compositions include unsubstituted β-cyclodextrin. In one or more embodiments, the pharmaceutical compositions include unsubstituted γ-cyclodextrin. In one or more embodiments, the pharmaceutical compositions includes a substituted α-cyclodextrin. In one or more embodiments, the pharmaceutical compositions include a substituted β-cyclodextrin. In one or more embodiments, the pharmaceutical compositions include a substituted γ-cyclodextrin.

In one or more embodiments, the pharmaceutical compositions of the present disclosure include hydroxypropyl-ρ-cyclodextrin (HP-3-CD; CAS No. 128446-35-5). In one or more embodiments, the pharmaceutical compositions of the present disclosure include hydroxypropyl-γ-cyclodextrin (HP-γ-CD; CAS No. 128446-34-3). In one or more embodiments, the pharmaceutical compositions of the present disclosure include methyl-β-cyclodextrin (M-β-CD; CAS No. 128446-36-6).

In one or more embodiments, the pharmaceutical compositions of the present disclosure include sulfobutylether-β-cyclodextrin (SBE-β-CD; CAS No. 182410-00-0). In one or more embodiments, the pharmaceutical compositions of the present disclosure include sodium sulfobutylether-β-cyclodextrin (Na-SBE-β-CD; CAS No. 182410-00-0). In one or more embodiments, the pharmaceutical compositions of the present disclosure include (SBE)₆₋₈-β-CD or Na-(SBE)₆₋₈-β-CD. Na-(SBE)₆₋₈-β-CD can be purchased as CAPTISOL from Ligand Pharmaceuticals Incorporated (San Diego, CA).

In one or more embodiments, the molar ratio or weight ratio of nervonic acid equivalents to the cyclodextrin in the pharmaceutical composition may be 0.01 part or greater, 0.1 part or greater, 1 part or greater, 2 parts or greater, 3 parts or grade, 4 parts or greater, 5 parts or greater, 6 parts or greater, 7 parts or greater, 8 parts or greater, 9 parts or greater, 10 parts or greater, 15 parts or greater, 20 parts or greater, 30 parts or greater, 40 parts or greater, or 50 parts or greater of the cyclodextrin for every one part nervonic acid equivalents. In one or more embodiments, the molar ratio or weight ratio of nervonic acid equivalents to the cyclodextrin in the pharmaceutical composition may be 100 parts or less, 50 parts or less, 40 parts or less, 30 parts or less, 20 parts of less, 15 parts or less, 10 parts or less, 9 parts or less, 8 parts or less, 7 parts or less, 6 parts or less, 5 parts or less, 4 parts or less, 3 parts or less, 2 parts or less, 1 part or less, or 0.1 part of the cyclodextrin for everyone one part nervonic acid equivalents.

In one or more embodiments, where the pharmaceutical composition is a solid (e.g., a solid dosage form) or a liquid (e.g., liquid dosage form) the weight percent (wt-%) of cyclodextrin compared to the total weight of the pharmaceutical composition may be 1 wt-% or greater, 5 wt-% or greater, 10 wt-% or greater, 15 wt-% or greater, 20 wt-% or greater, 25 wt-% or greater, 35 wt-% or greater, 40 wt-% or greater, 45 wt-% or greater, 50 wt-% or greater, 55 wt-% or greater, 60 wt-% or greater, 65 wt-% or greater, 70 wt-% or greater, or 75 wt-% or greater. In one or more embodiments, the weight percent (wt-%) of cyclodextrin compared to the total weight of the pharmaceutical composition may be 80 wt-% or less, 75 wt-% or less, 70 wt-% or less, 65 wt-% or less, 60 wt-% or less, 55 wt-% or less, 50 wt-% or less, 45 wt-% or less, 40 wt-% or less, 35 wt-% or less, 30 wt-% or less, 25 wt-% or less, 20 wt-% or less, 15 wt-% or less, or 5 wt-% or less.

In one or more embodiments, where the pharmaceutical composition a liquid (e.g., liquid dosage form) the weight percent of cyclodextrin compared to volume percent of the composition (%-(w/v)) may be 1% (w/v) or greater, 5% (w/v) or greater, 10% (w/v) or greater, 15% (w/v) or greater, 20% (w/v) or greater, 25% (w/v) or greater, 35% (w/v) or greater, 40% (w/v) or greater, 45% (w/v) or greater, 50% (w/v) or greater, 55% (w/v) or greater, 60% (w/v) or greater, 65% (w/v) or greater, 70% (w/v) or greater, 75% (w/v) or greater, 80% (w/v) or greater, 90% (w/v) or greater, 100% (w/v) or greater, 110% (w/v) or greater, or 120% (w/v) or greater. In one or more embodiments, the weight percent of cyclodextrin compared to volume percent of the composition may be 150% (w/v) or less, 120% (w/v) or less, 110% (w/v) or less, 100% (w/v) or less, 90% (w/v) or less, 80% (w/v) or less, 75% (w/v) or less, 70% (w/v) or less, 65% (w/v) or less, 60% (w/v) or less, 55% (w/v) or less, 50% (w/v) or less, 45% (w/v) or less, 40% (w/v) or less, 35% (w/v) or less, 30% (w/v) or less, 25% (w/v) or less, 20% (w/v) or less, 15% (w/v) or less, or 5% (w/v) or less.

In one or more embodiments, where the pharmaceutical composition is a liquid (e.g., a liquid dosage form) the concentration of the cyclodextrin in the pharmaceutical composition may be 0.01 mg/ml or greater, 0.05 mg/ml or greater, 0.1 mg/ml or grater, 0.5 mg/ml or greater, 1 mg/ml or greater 1.5 mg/ml or greater, 2 mg/ml or greater, 2.5 mg/ml or greater, 3 mg/ml or greater, 3.5 mg/ml or greater, 4 mg/ml or greater, 4.5 mg/ml or greater, 5 mg/ml or greater, 5.5 mg/ml or greater, 6 mg/ml or greater, 6.5 mg/ml or greater, 7 mg/ml or greater, 7.5 mg/ml or greater, 8 mg/ml or greater, 8.5 mg/ml or greater, 9 mg/ml or greater, 9.5 mg/ml or greater, 10 mg/ml or greater, 12.5 mg/ml or greater, 15 mg/ml or grater, 17.5 mg/ml or greater, 20 mg/ml or greater, 25 mg/ml or greater, 30 mg/ml or greater, or 40 mg/ml or greater, 50 mg/ml or greater, 75 mg/ml or greater, 100 mg/ml or greater, 125 mg/ml or greater, 150 mg/ml or greater, 175 mg/ml or greater, 200 mg/ml or greater, or 250 mg/ml or greater. In one or more embodiments, the concentration of the cyclodextrin in the pharmaceutical composition may be 300 mg/ml or less, 250 mg/ml or less, 200 mg/ml or less, 175 mg/ml or less, 150 mg/ml or less, 125 mg/ml or less, 100 mg/ml or less, 75 mg/ml or less, 50 mg/ml or less, 40 mg/ml or less, 30 mg/ml or less, 25 mg/ml or less, 20 mg/ml or less, 17.5 mg/ml or less, 15 mg/ml or less, 12.5 mg/ml or less, 10 mg/ml or less, 9.5 mg/ml or less, 9 mg/ml or less, 8.5 mg/ml or less, 8 mg/ml or less, 7.5 mg/ml or less, 7 mg/ml of or less, 6.5 mg/ml or less, 6 mg/ml or less, 5.5 mg/ml or less, 5 mg/ml or less, 4.5 mg/ml or less, 4 mg/ml or less, 3.5 mg/ml or less, 3 mg/ml or less, 2.5 mg/ml or less, 2 mg/ml or less, 1.5 mg/ml or less, 1 mg/ml or less, 0.5 mg/ml or less, 0.1 mg/ml or less, or 0.05 mg/ml or less.

In one or more embodiments, where the pharmaceutical composition is a liquid (e.g., a liquid dosage form) the concentration of the cyclodextrin in the pharmaceutical composition may be 0.000001 M or greater, 0.00001 M or greater, 0.0001 M or greater, 0.001 M or greater, 0.01 M or greater, 0.1 M or greater, 1 M or greater, or 5 M or greater. In one or more embodiments, the concentration of the cyclodextrin in the pharmaceutical composition may be 10 M or less, 5 M or less, 1 M or less, 0.1 M or less, 0.01 M or less, 0.001 M or less, 0.0001 M or less, 0.00001 or less, or 0.000001 M or less.

Table 1 shows example pharmaceutical composition formulations that have been tested (see Example for how the formulations were made).

TABLE 1

| | | | | | | Second | | |
|---|---|---|---|---|---|---|---|---|
| | NA | | Cyclodextrin | Water | | solvent | | |
| | amount | | amount | amount | Second | amount | buffering | |
| No. | (mg) | Cyclodextrin Used | (% w/v) | (μL) | solvent | (μL) | agent | pH |
| 1 | 2 | sulfobutylether-β- | 44 | 900 | — | — | — | — |
| | | cyclodextrin | | | | | | |
| 2 | 2 | sulfobutylether-β- | 44 | 1000 | — | — | PBS | — |
| | | cyclodextrin | | | | | | |
| 3 | 2 | sulfobutylether-β- | 44 | 900 | DMSO | 100 | — | — |
| | | cyclodextrin | | | | | | |
| 4 | 2 | sulfobutylether-β- | 44 | 900 | ethanol | 100 | — | — |
| | | cyclodextrin | | | | | | |
| 5 | 2 | sulfobutylether-β- | 40 | 900 | ethanol | 100 | — | — |
| | | cyclodextrin | | | | | | |
| 6 | 2 | sulfobutylether-β- | 20 | 900 | ethanol | 100 | — | — |
| | | cyclodextrin | | | | | | |
| 7 | 5 | sulfobutylether-β- | 44 | 900 | ethanol | 100 | — | — |
| | | cyclodextrin | | | | | | |
| 8 | 7.5 | sulfobutylether-β- | 44 | 900 | ethanol | 100 | — | — |
| | | cyclodextrin | | | | | | |
| 9 | 10 | sulfobutylether-β- | 44 | 900 | ethanol | 100 | — | — |
| | | cyclodextrin | | | | | | |
| 10 | 5 | hydroxypropyl-β- | 20 | 1000 | — | — | — | — |
| | | cyclodextrin | | | | | | |
| 11 | 5 | sulfobutylether-β- | 20 | 1000 | — | — | — | — |
| | | cyclodextrin | | | | | | |
| 12 | 5 | γ-cyclodextrin | 10 | 1000 | — | — | PBS | 7-8 |
| 13 | 5 | γ-cyclodextrin | 10 | 750 | ethanol | 250 | PBS | 7-8 |
| 14 | 5 | hydroxypropyl-γ- | 20 | 1000 | — | | PBS | 7-8 |
| | | cyclodextrin | | | | | | |
| 15 | 5 | hydroxypropyl-γ- | 20 | 750 | ethanol | 250 | PBS | 7-8 |
| | | cyclodextrin | | | | | | |

In another aspect, the present disclosure describes methods of treating a disease by administering to the subject a pharmaceutical composition of the present disclosure. In one or more embodiments, the subject is a human. The pharmaceutical compositions of the present disclosure may be used to treat subjects having or at risk of having adrenoleukodystrophy (ALD), a neurological disorder, or both. The pharmaceutical compositions of the present disclosure may be used to treat subjects having or at risk of having various phenotypes of adrenoleukodystrophy (ALD) including Addison's disease, cerebral ALD, adrenomyeloneuropathy, asymptomatic ALD, or any combination thereof. The subject having or at risk of having ALD may be a female or a male. Example neurological disorders that may be prevented and/or treated using the pharmaceutical compositions of the present disclosure include, but are not limited to, Parkinson's Disease and Alzheimer's Disease.

“Treat” or variations thereof refer to reducing, limiting progression, ameliorating, or resolving, to any extent, the symptoms or signs related to a condition. “Treating” or a “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject.

Treating a condition can be prophylactic or, alternatively, can be initiated after the subject exhibits one or more symptoms or clinical signs of ALD and/or a neurological disease. Treatment that is prophylactic—e.g., initiated before a subject manifests a symptom or clinical sign of ALD and/or a neurological disease is referred to herein as treatment of a subject that is “at risk” of having ALD and/or a neurological disease. As used herein, the term “at risk” refers to a subject that may or may not actually possess the described risk. As another example, a subject “at risk” is a subject possessing one or more risk factors associated with ALD and/or a neurological disease such as, for example, genetic predisposition or medical history. Additionally, an at risk subject may have mutation in a gene associated with ALD (e.g., ABCD1 gene) but may not exhibit symptoms of ALD. Treatment may also be continued after symptoms have resolved, for example to prevent or delay their recurrence.

Accordingly, a pharmaceutical composition can be administered before, during, or after the subject first exhibits a symptom or clinical sign of ALD and/or a neurological disease. Treatment initiated before the subject first exhibits a symptom or clinical sign associated with ALD and/or a neurological disease may result in decreasing the likelihood that the subject experiences clinical evidence of ALD and/or a neurological disease compared to a subject to which the composition is not administered, decreasing the severity of symptoms and/or clinical signs of ALD and/or a neurological disease, and/or completely resolving ALD. Treatment initiated after the subject first exhibits a symptom or clinical sign associated with ALD may result in decreasing the severity of symptoms and/or clinical signs of ALD compared to a subject to which the composition is not administered, and/or completely resolving the ALD.

Thus, the method includes administering an effective amount of a pharmaceutical composition described herein to a subject having, or at risk of having, ALD and/or a neurological disease. In this aspect, an “effective amount” is an amount effective to reduce, limit progression, ameliorate, or resolve, to any extent, a symptom or clinical sign related to ALD and/or a neurological disease. The dosage ranges for the administration of the pharmaceutical compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex, and extent of the disease in the patient.

Pharmaceutical compositions described herein, can be administered in any suitable manner. In one or more embodiments where the pharmaceutical composition is a drug product and the drug product is in a solid dosage form, the pharmaceutical composition can be administered orally. In one or more embodiments where the pharmaceutical composition is a drug product and the drug product is in a liquid dosage form, the pharmaceutical composition can be administered orally. In other embodiments where the pharmaceutical composition is a drug product and the drug product is in a liquid dosage form, the pharmaceutical composition can be administered intravenously, subcutaneously, intrathecally, intramuscularly, or intraperitoneally by infusion or injection.

Doses and dosing regimens may vary based on the age, sex, weight, and disease state of the subject. One can alter the dosages and/or frequency as needed to achieve a desired level of nervonic acid equivalents. Accordingly, it is not practical to set forth generally the amount that constitutes an amount of nervonic acid equivalents effective for all possible applications and/or single doses. Those of ordinary skill in the art, however, can readily determine the appropriate dosing amount with due consideration of such factors.

The absolute amount of nervonic acid equivalents administered to the subject may or may not be the same absolute amount of nervonic acid equivalents provided in the pharmaceutical compositions of the present disclosure. Pharmaceutical compositions provided as drug products may be administered without modifying the drug product to alter the dose. For example, a solid dosage unit form may be administered to the patient as a whole. Pharmaceutical composition provided as drug products may be modified before or during administration to a subject in order to administer the desired dose of nervonic acid equivalents. For example, a solid dosage unit form may be split into a smaller dose. Additionally, a liquid dosage form may be diluted prior to injection or infusion.

In one or more embodiments, the method can include administering sufficient nervonic acid equivalents to provide a dose of, for example, from about 100 ng/kg to about 50 mg/kg to the subject (e.g., 30 mg/kg), although in one or more embodiments the methods may be performed by administering nervonic acid equivalent in a dose outside this range. In one or more of these embodiments, the method includes administering sufficient nervonic acid equivalents to provide a dose of from about 1 mg/kg to about 50 mg/kg to the subject, for example, a dose of from about 1 mg/kg to about 30 mg/kg.

A single dose may be administered all at once, continuously for a prescribed period of time, or in multiple discrete administrations. When multiple administrations are used, the amount of each administration may be the same or different. For example, a dose of 1 mg per day may be administered as a single administration of 1 mg, continuously over 24 hours, as two or more equal administrations (e.g., two 0.5 mg administrations), or as two or more unequal administrations (e.g., a first administration of 0.75 mg followed by a second administration of 0.25 mg). When multiple administrations are used to deliver a single dose, the interval between administrations may be the same or different.

In one or more embodiments, the active agent may be administered, for example, from a single dose to multiple doses per week or multiple doses per day, although in one or more embodiments the method can involve a course of treatment that includes administering doses of the active agent at a frequency outside this range. When a course of treatment involves administering multiple doses within a certain period, the amount of each dose may be the same or different. For example, a course of treatment can include an initial loading dose, followed by a maintenance dose that is lower than the loading dose. Also, when multiple doses are used within a certain period, the interval between doses may be the same or be different. In certain embodiments, pharmaceutical composition may be administered daily or multiple times per day.

A single dose may be administered all at once, continuously for a prescribed period of time, or in multiple discrete administrations. When multiple administrations are used, the amount of each administration may be the same or different. For example, a dose of 1 mg per day may be administered as a single administration of 1 mg, continuously over 24 hours, as two or more equal administrations (e.g., two 0.5 mg administrations), or as two or more unequal administrations (e.g., a first administration of 0.75 mg followed by a second administration of 0.25 mg). When multiple administrations are used to deliver a single dose, the interval between administrations may be the same or different.

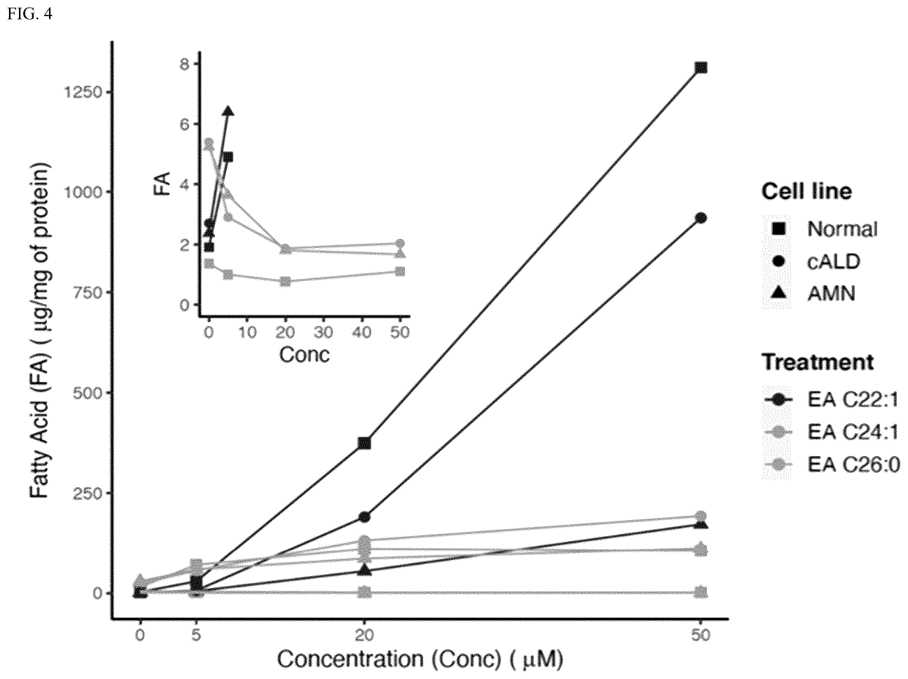

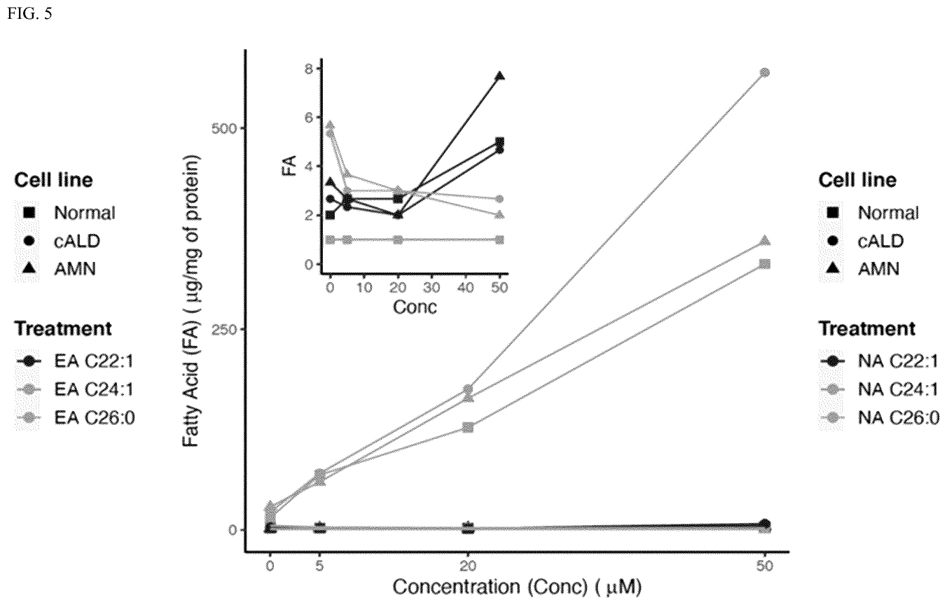

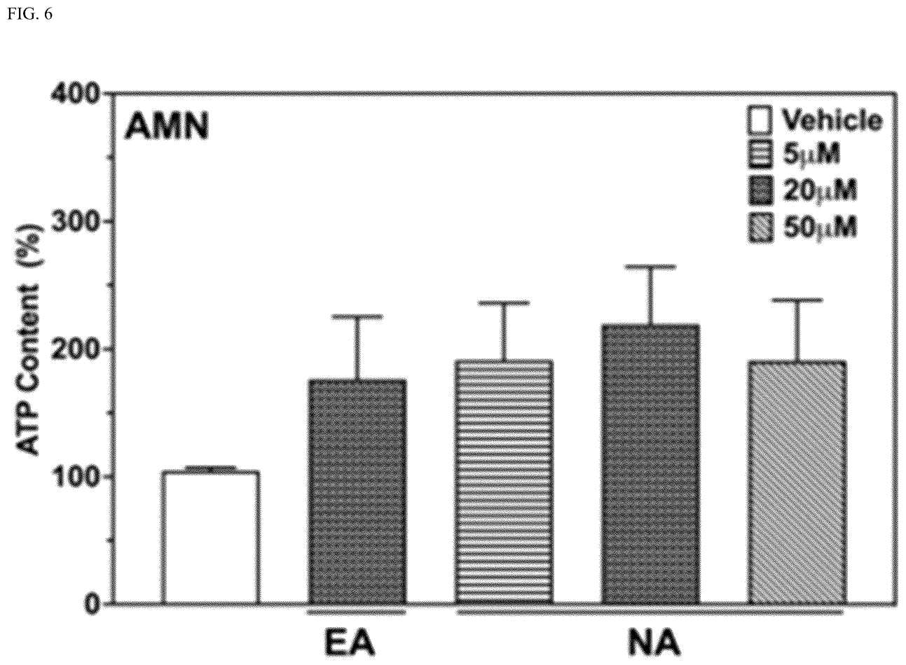

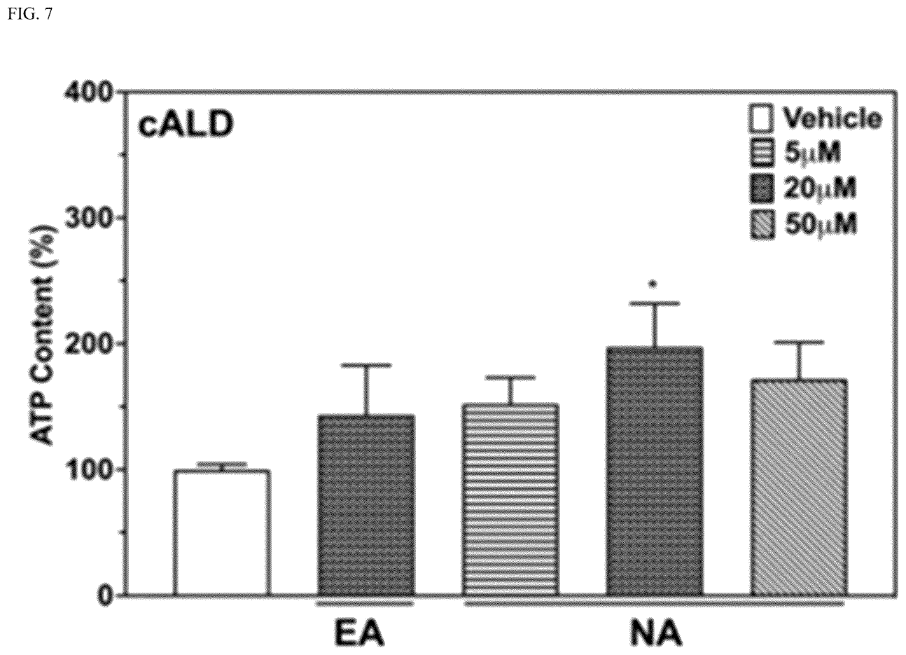

In vitro and in vivo experiments were done to evaluate the ability of nervonic acid to modulate ALD associated biochemistry in various ALD phenotypes including childhood cerebral ALD (cALD) and adrenomyeloneuropathy (AMN). Nervonic acid (NA) was compared to the erucic acid (EA), the active ingredient in Lorenzo's oil. Initial experiments revealed that NA was able to decrease the amount of free and complex C26:0 fatty acids as well as increase the amount of ATP in ALD cells. More specifically, examination of cells treated with varying amounts of NA and EA revealed that NA had a dose-dependent effect on decreasing the amount of C26:0 fatty acid in AMN and cALD cells (See FIG. 4 and FIG. 5; see also Terluk et al., Neurotherapeutics 2022 April; 19(3):1007-1017. doi: 10.1007/s13311-022-01226-7). Additionally, the potential of NA and EA to alter the basal energy production measured as ATP content in ALD cells was assessed (Terluk et al., Neurotherapeutics 2022 April; 19(3):1007-1017. doi: 10.1007/s13311-022-01226-7). Following a five-day treatment with NA, cellular ATP content was increased in cALD cells (FIG. 6). This effect was significant compared to vehicle-treated cells at 20 μM nervonic acid, while a trend was observed at other concentrations in cALD and AMN cells (FIG. 6 and FIG. 7). A similar concentration of erucic acid (20 μM) did not show a marked increase in energy production indicating nervonic acid to be functionally beneficial in addition to its direct biochemical effects.

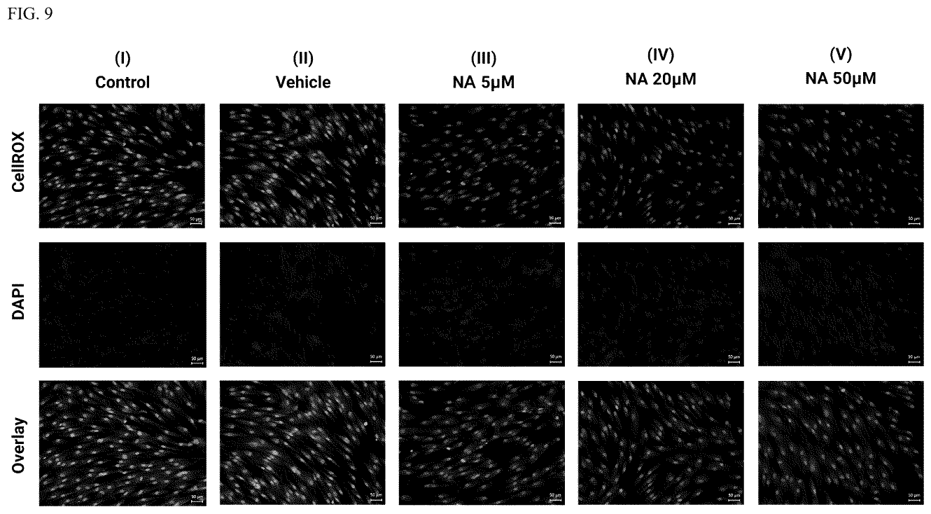

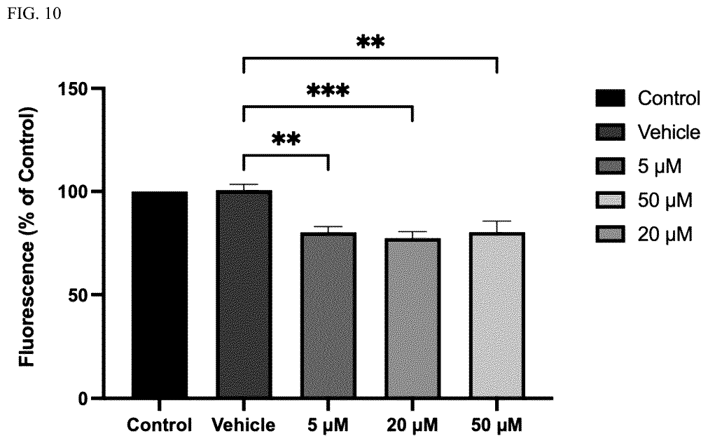

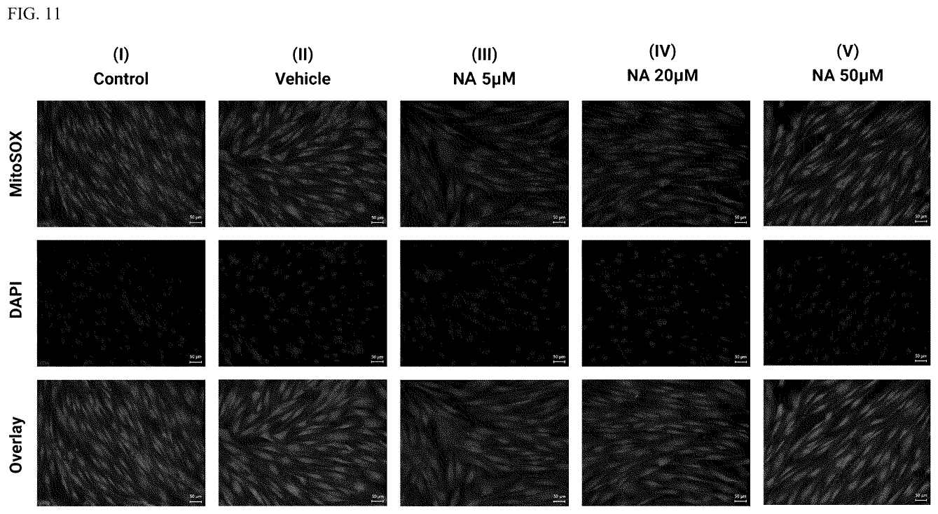

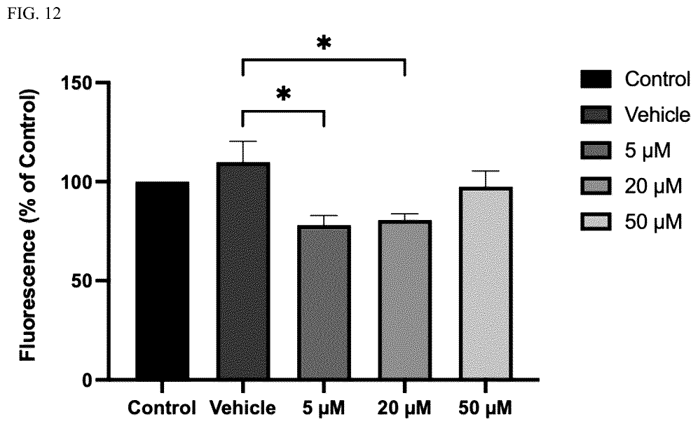

A second set of in vitro experiments were conducted to more thoroughly understand the mechanism of action of nervonic acid in the mitochondria of ALD cells. AMN patient derived fibroblasts were treated with various concentrations of NA (5 μM, 20 μM, and 50 μM in ethanol) and evaluated for mitochondrial oxidative stress by evaluating reactive oxygen species (see Example) and mitochondrial respiration (see Example). FIG. 9 and FIG. 11 show images of the NA AMN treated cells stained for reactive oxygen species (green) or mitochondrial superoxide (red) and nuclei (blue; see Imaging Study in Example). FIG. 10 and FIG. 12 show bar graphs quantifying the reactive oxygen species fluorescence of FIG. 9 and the mitochondrial superoxide fluorescence of FIG. 11. Compared to the vehicle and control, treatment with 5 μM, 20 μM, or 50 μM NA resulted in a decrease in reactive oxygen species (FIG. 9 and FIG. 10) and treatment with 5 μM, or 20 μM NA resulted in a decrease in mitochondrial superoxide (FIG. 11 and FIG. 12).

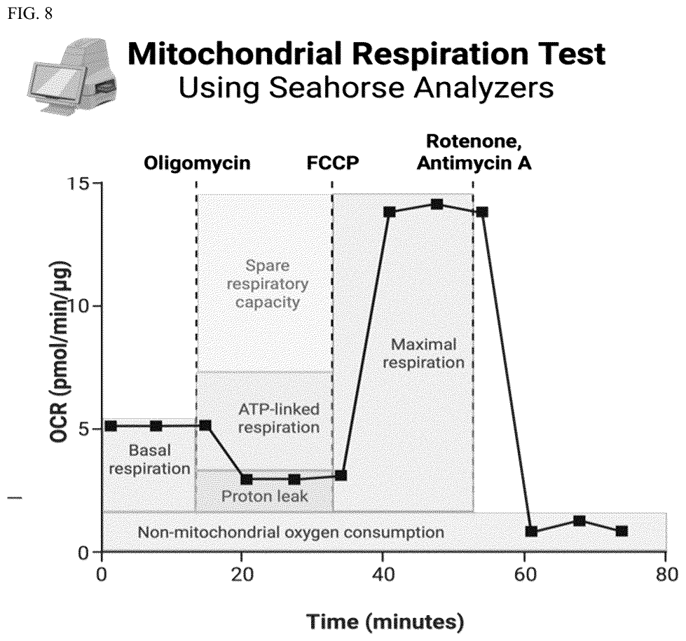

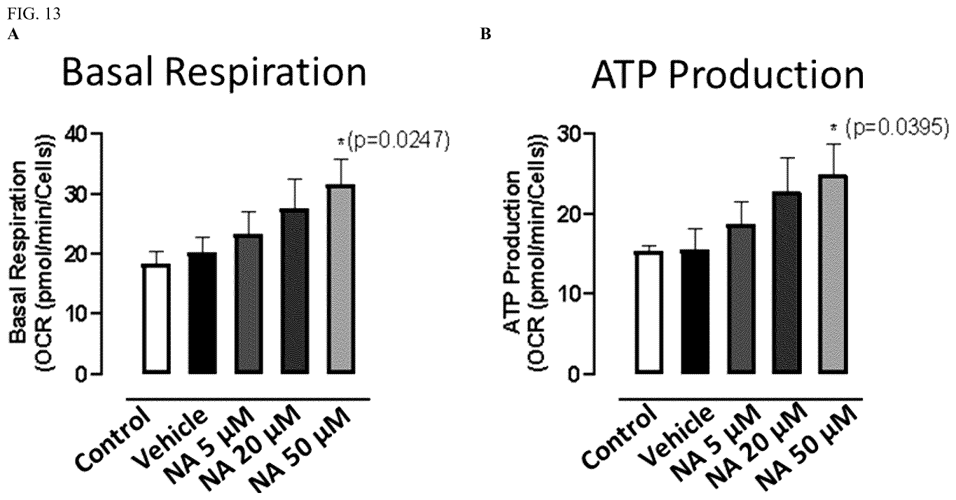

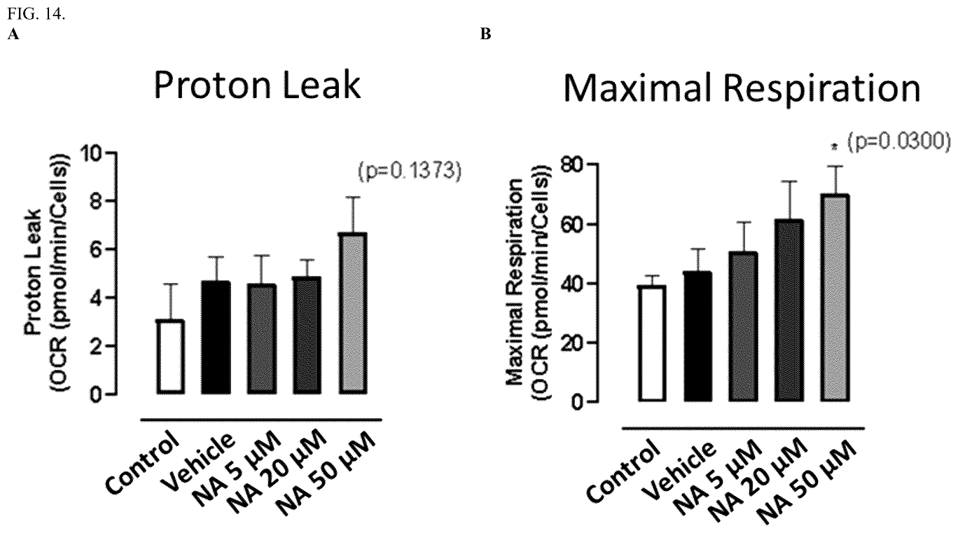

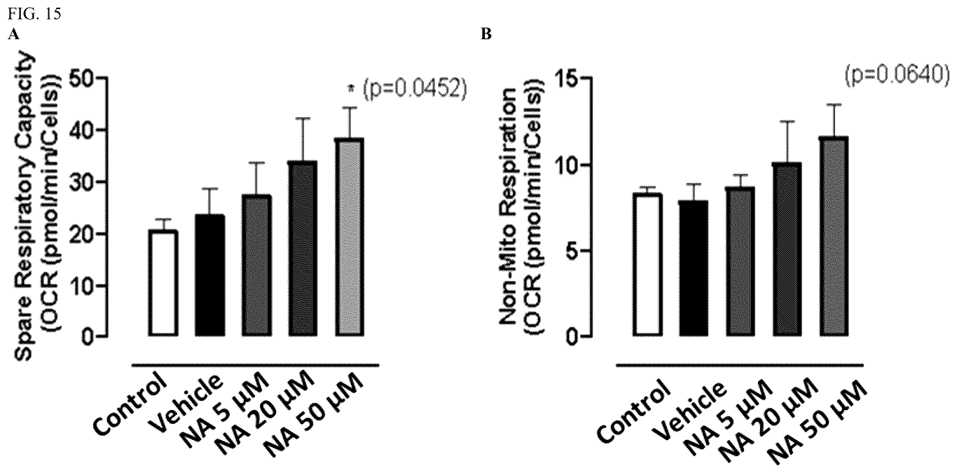

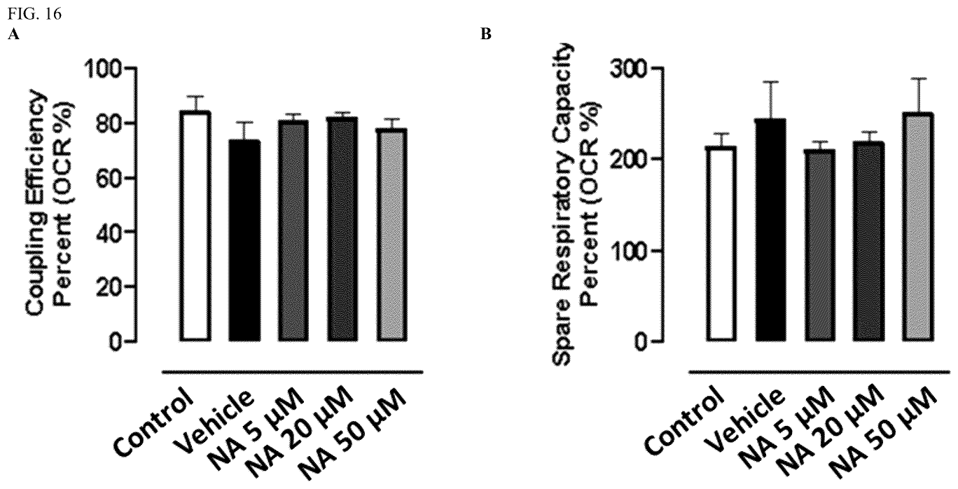

Mitochondrial function was assessed using a cell mitochondrial stress test (MST; see Seahorse study in the Example). As depicted in FIG. 8, in this experiment, inhibitors of various electron transport chain components (oligomycin; FCCP/carbonyl cyanide 4-(trifluoromethoxy)phenylhydrazone; rotenone and antimycin A) are sequentially incubated with cells and the oxygen consumption rate (OCR) measured following each incubation to evaluate various individual parameters of respiration. The OCR of cells that were treated with NA was compared with cells not treated with NA. FIG. 13A-13, FIG. 14A-B, FIG. 15A-B, and FIG. 16A-B are plots showing the results of the respiration test. Treatment with NA resulted in an increase in basal respiration, ATP production, maximal respiration, and spare respiratory capacity.

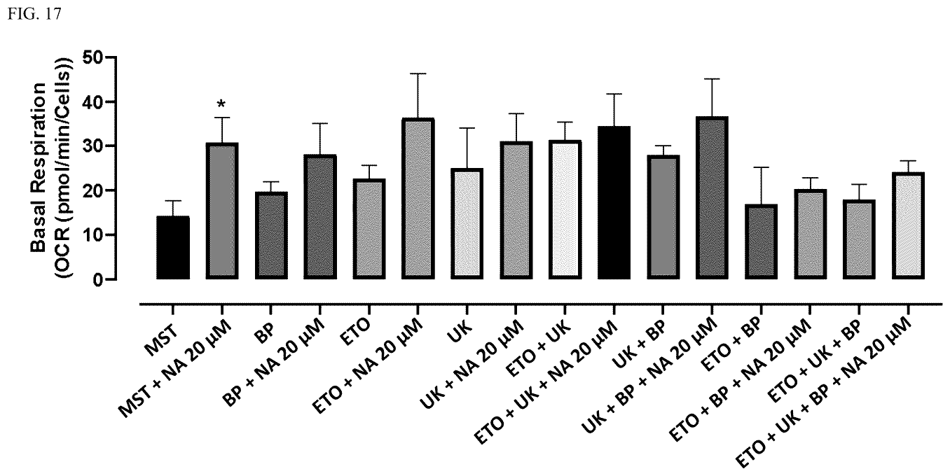

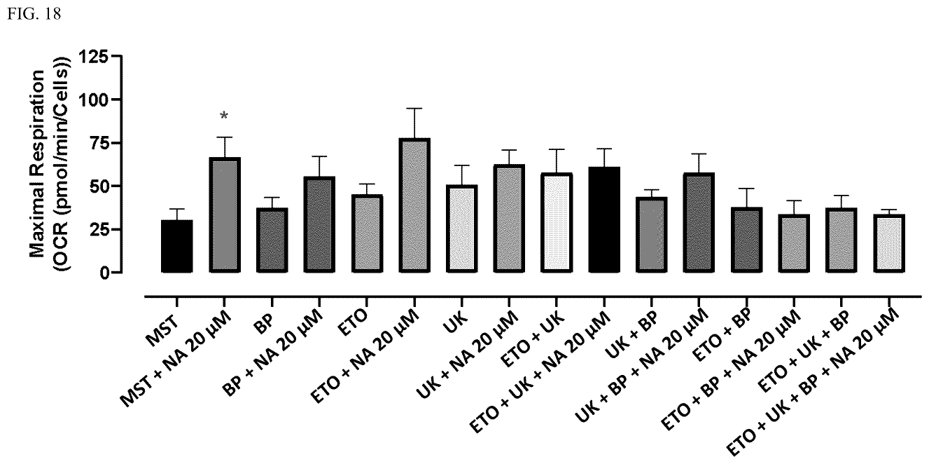

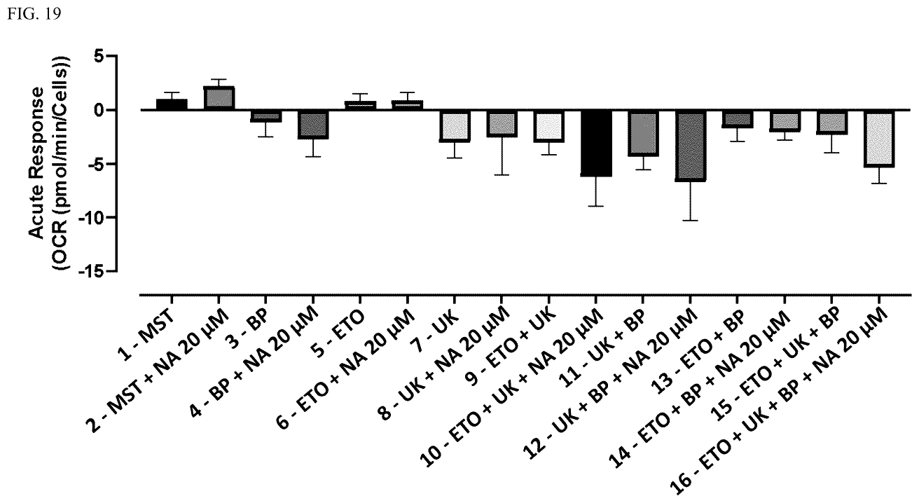

A long chain fatty acid (LCFA) oxidation stress test (SEAHORSE XF assay; Agilent Technologies, Inc., Santa Clara, CA) was used to assess how NA affects mitochondrial respiration when various metabolic pathways of mitochondria are inhibited. Specific inhibitors to block glucose, fatty acid or glutamine pathways were used alone or in combination to understand the substrate specific effects of NA. In this assay the mitochondrial stress test (MST) is repeated with one or more inhibitors of glycolysis, the TCA cycle, electron transport, and oxidative phosphorylation pathways are present. Etomoxir (Eto) is used to inhibit carnitine palmitoyl transferase 1a (CPT1a), an enzyme important in the mechanism for beta oxidation (degradation) of long chain fatty acids. UK5099 (UK) is used to inhibit transport of glucose and/or pyruvate by targeting the mitochondrial pyruvate carrier (MPC). BPTES (BP) is used to inhibit glutaminase GLS1, an enzyme that converts glutamine into glutamate, a substrate for further oxidation in the TCA cycle. AMN cells were incubated with one or more of the inhibitors with or without 20 μM NA and run through the mitochondrial stress test. The results are shown in FIG. 17, FIG. 18, and FIG. 19. No significant change in basal respiration (FIG. 17), maximal respiration (FIG. 18), and acute response (FIG. 19) was observed for any of the conditions tested except in the mitochondrial stress test (MST) where the oxidative stress test inhibitors were not present.

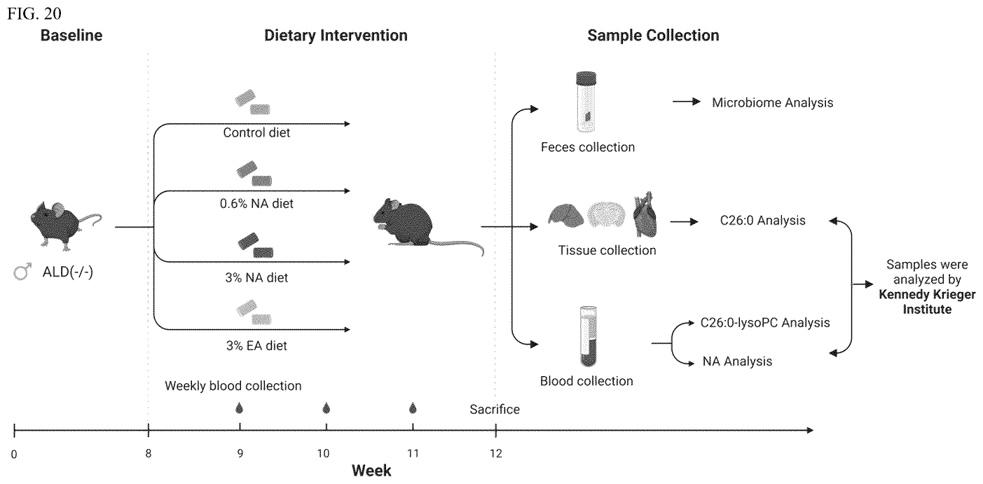

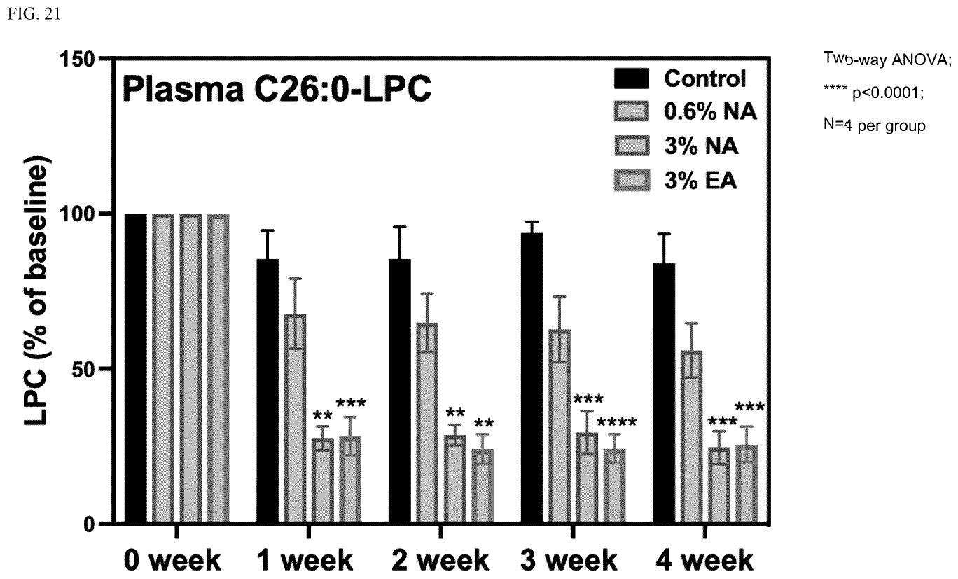

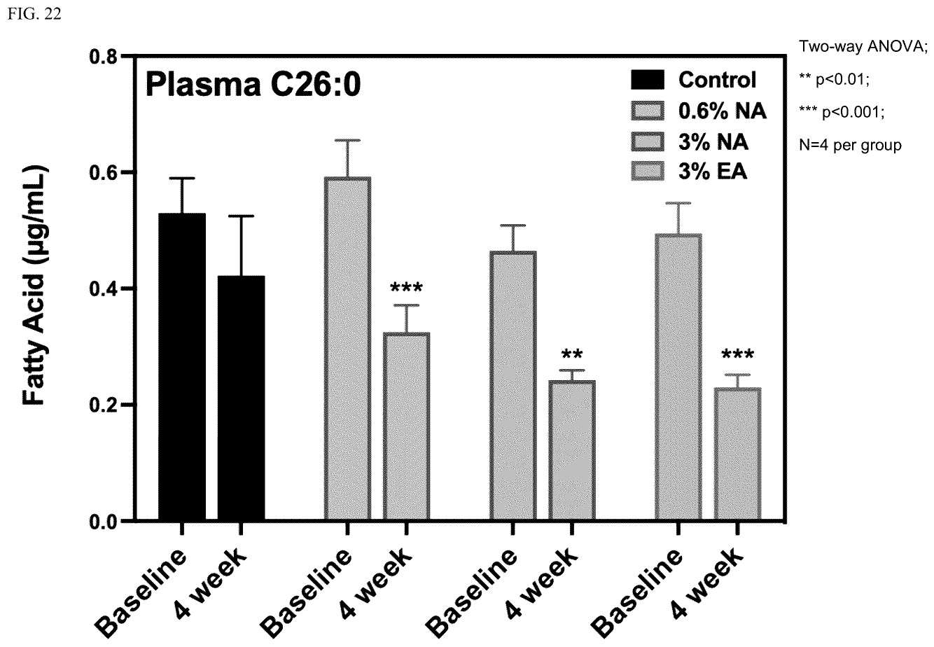

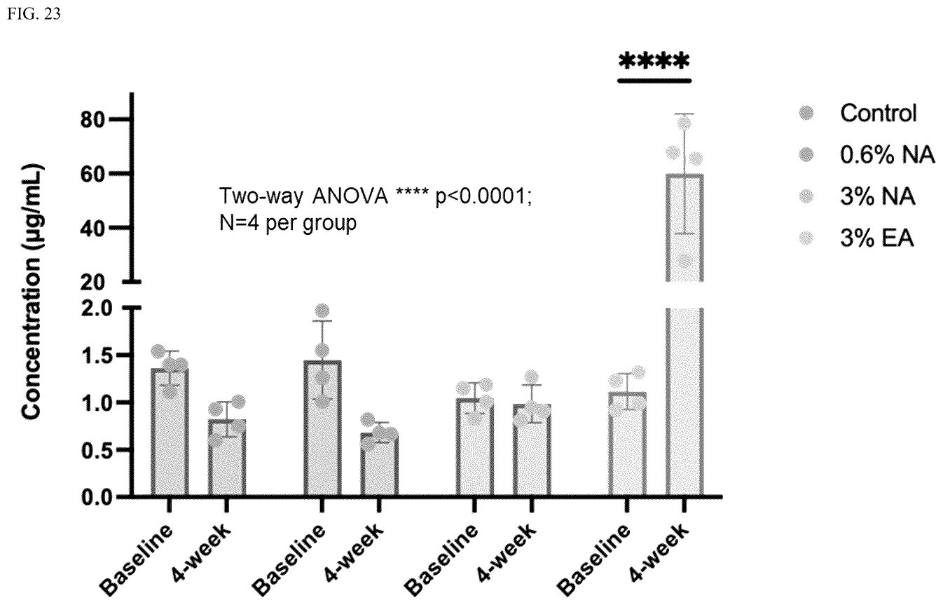

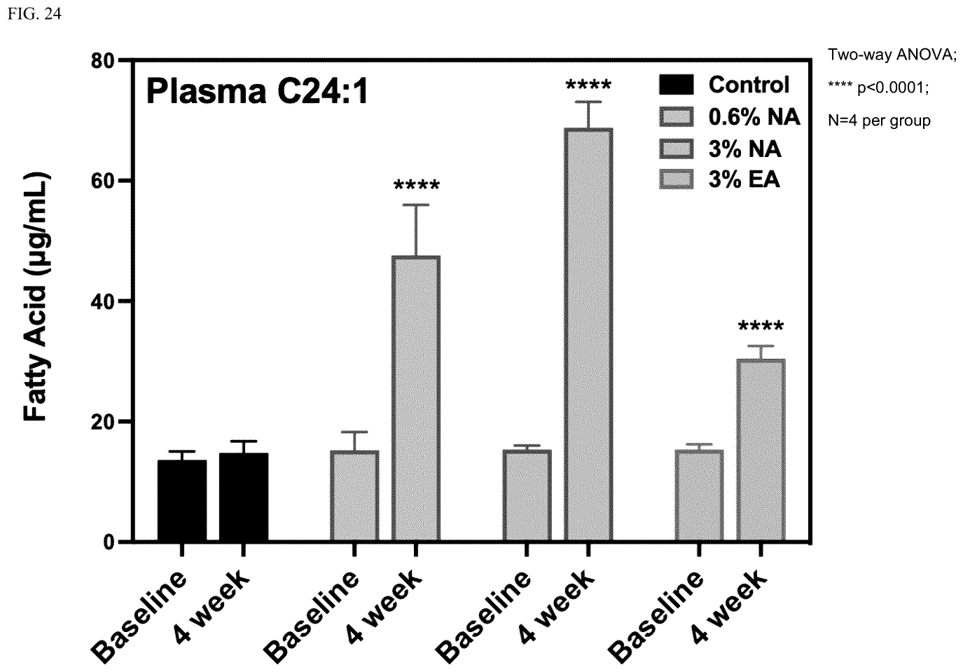

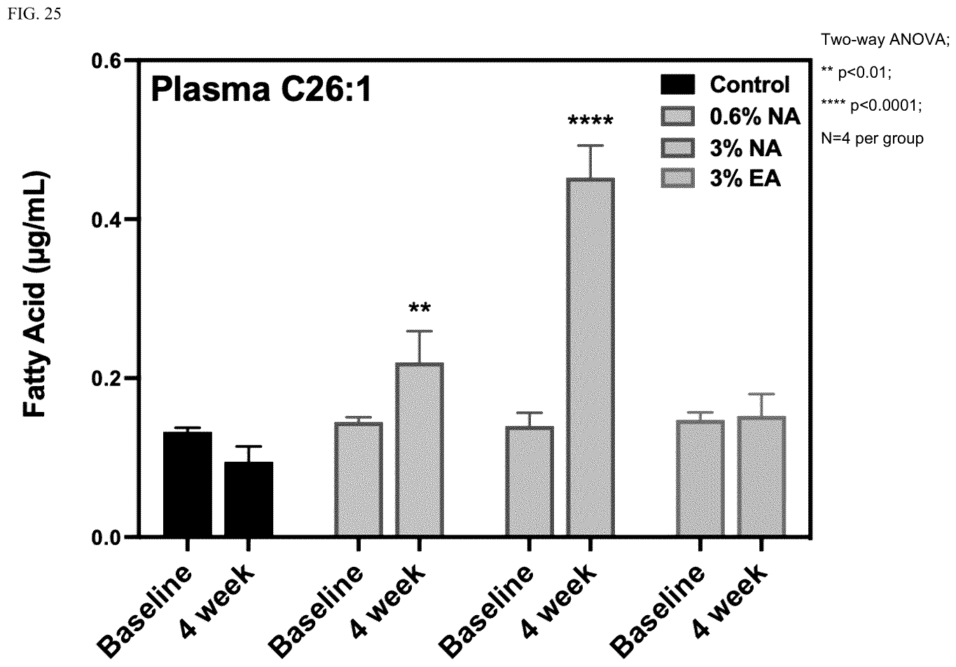

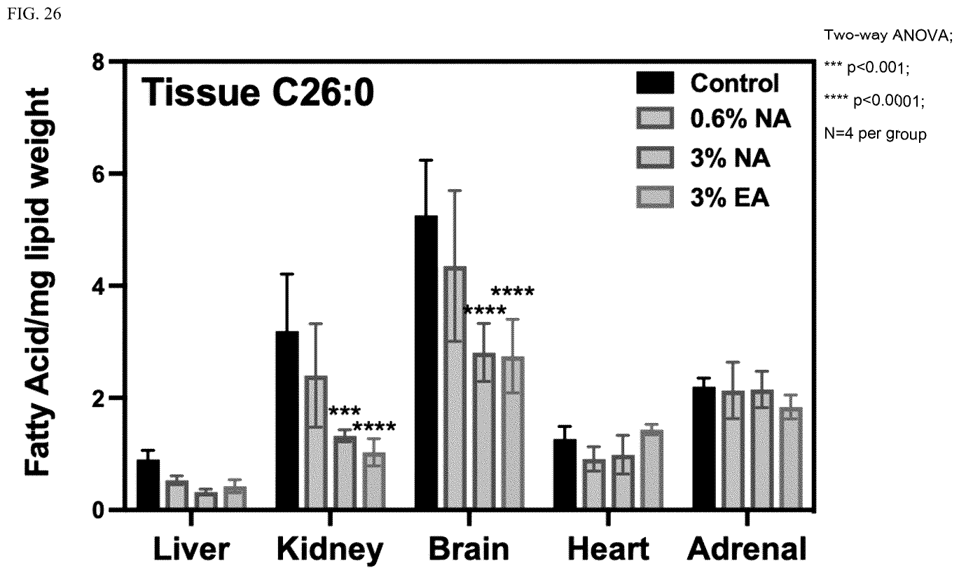

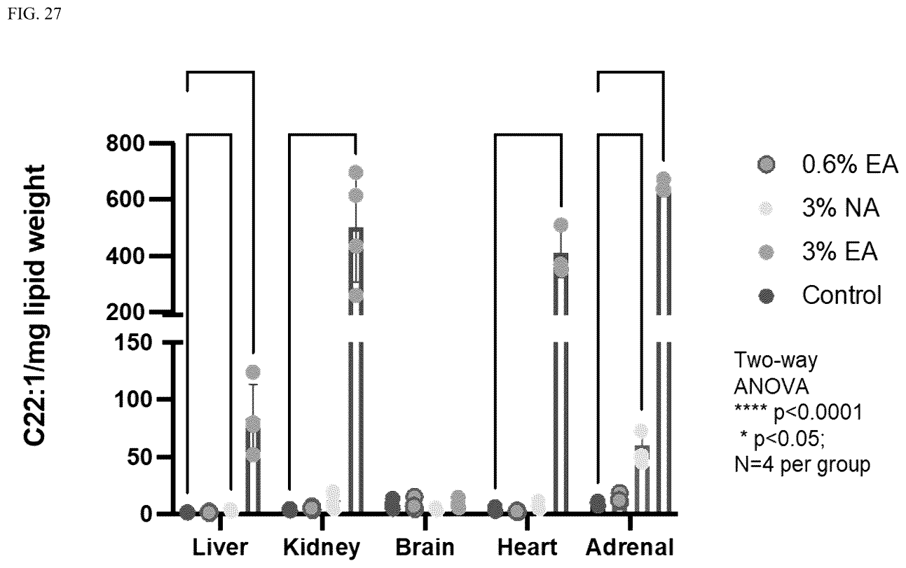

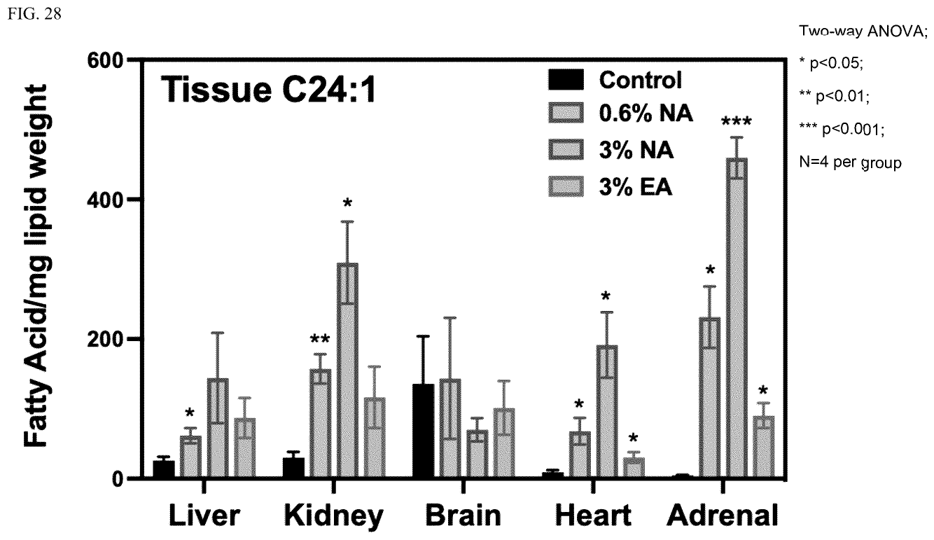

An in vivo ALD double knockout mice model was used to evaluate the effect of NA or EA on various fatty acid concentrations in the tissue of the mice see Example). Mice were fed a diet containing 0.6% NA, 3% NA, or 3% EA for four weeks (FIG. 20). Blood samples taken every week and tissues harvested after four weeks of treatment were analyzed for fatty acid concentration. All treatment groups showed a decrease in the amount of C26:0-lysophsphatidylcholine (LPC) in blood of the mice at all weeks tested (FIG. 21). Additionally, NA showed a dose dependent decrease in plasma C26:0 (FIG. 21 and FIG. 22). A significant increase in the level of C22:1 (EA) was observed in the plasma only after a 4-week treatment with 3% EA (FIG. 23). All treatment groups showed a significant increase in plasma C24:1 (NA) levels (FIG. 24). Intake of NA caused a significant increase in C26:1 levels indicating rerouting of fatty acid metabolism (FIG. 25). Treatment with NA resulted in a decrease in the C26:0 levels in tissues (FIG. 26). Additionally, no significant difference in the levels of C26:0 in tissues was observed between the 3% NA treatment group and the 3% EA treatment group (FIG. 26). Treatment with EA resulted in a significant elevation in the levels of C22:1 (EA) in the liver, kidney, heart, and adrenal compared to the control group (FIG. 27). The brain did not show an elevation in the amount of C22:1 (FIG. 27). Similar to treatment with EA, treatment with NA led to a significant elevation in the levels of C24:1 in the liver, kidney, heart, and adrenal gland compared to the control group (FIG. 28).

The in vivo data revealed that NA significantly reduced the plasma C26:0 Lyso-phosphatidylcholine (C26:0 LPC) in a dose-dependent manner in ALD mice after 4-week treatment and can show decrease in other complex lipids, such as ceramides. However, ALD mice tend to show variability in plasma (26:0 levels (FIG. 22). However, C26:0-LPC is more reliable than C26:0 and as such, C26:0-LPC may be a useful marker in screening. The in vivo data also revealed that the accumulation of C26:0 in different tissues is comparable in 3% NA and 3% EA treatment groups. Finally, following four-week treatment, NA levels reached a steady state and remained at approximately 70 pig/mL in plasma.

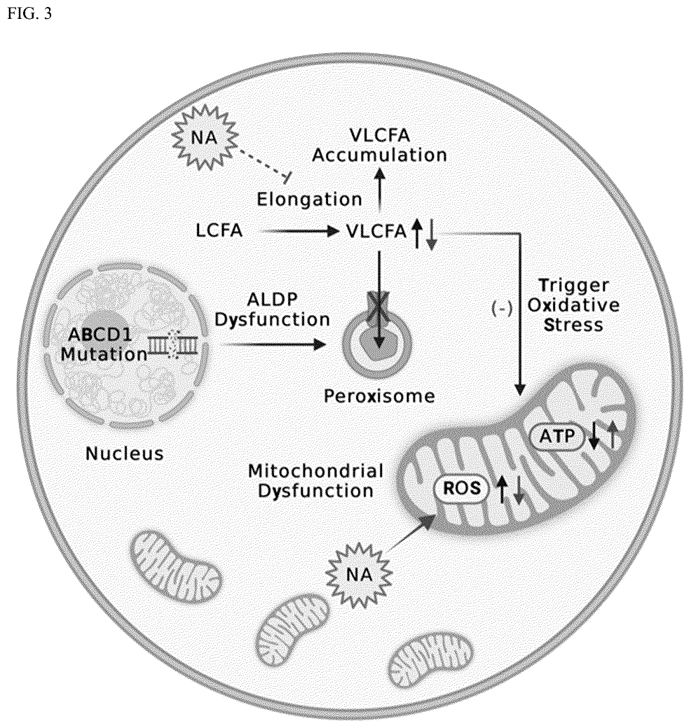

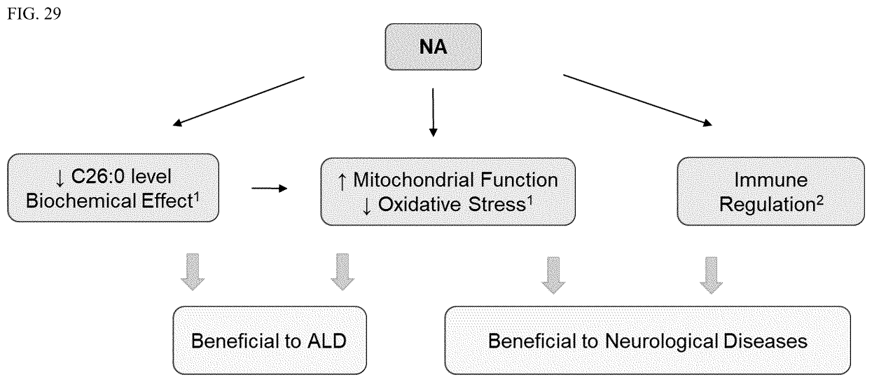

FIG. 3 and FIG. 29 outline the hypothesized mechanism of action and biochemical effects of NA on ALD cells. Clinically relevant concentrations of NA may have therapeutic efficacy by targeting the very long chain fatty acids (VLCFA) pathology in ALD and protecting from cellular oxidative stress in neurological disorders. More specifically, NA is able to decrease the C26:0 levels, increase mitochondrial function, and decrease mitochondrial oxidative stress. Further, NA may affect immune regulation.

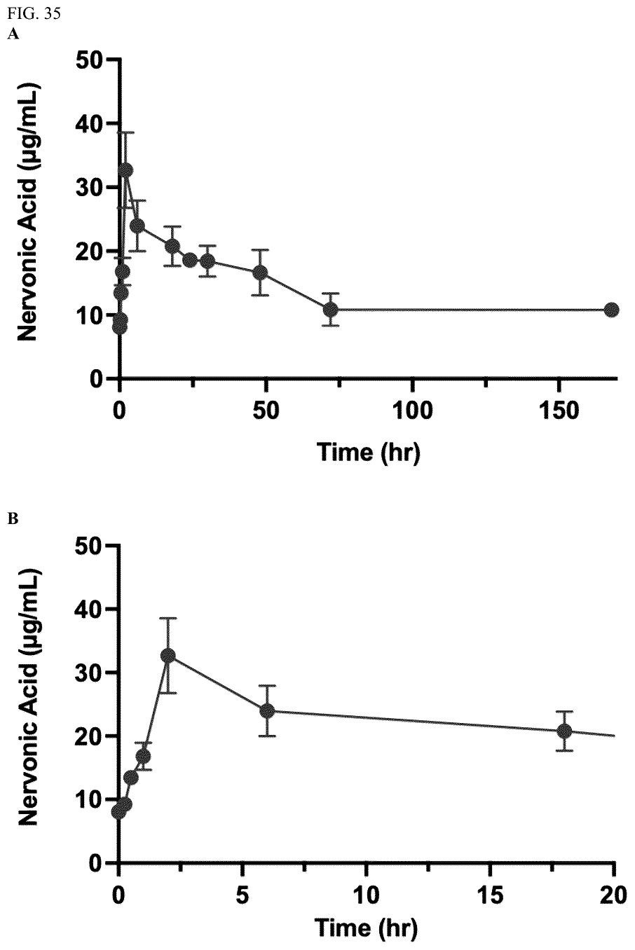

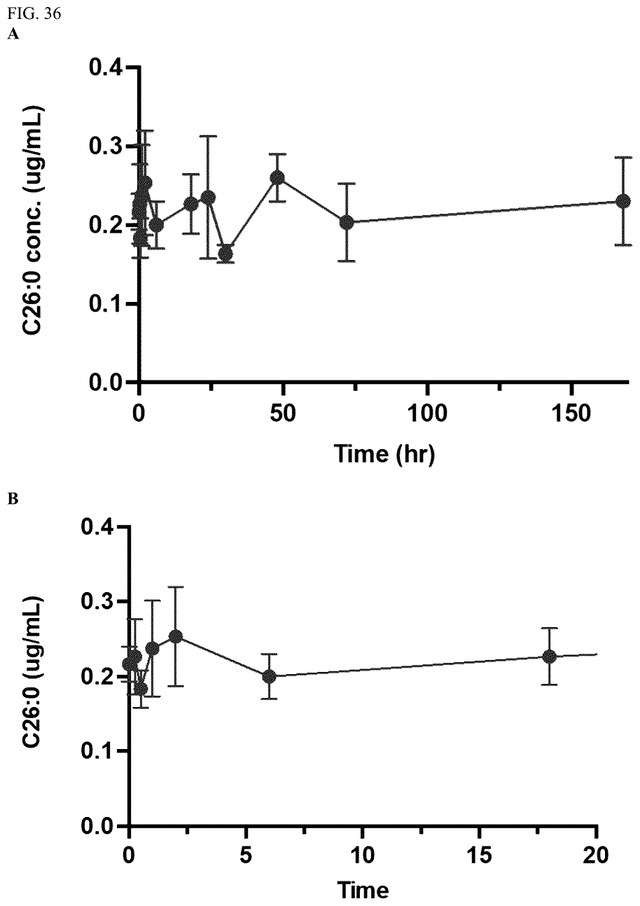

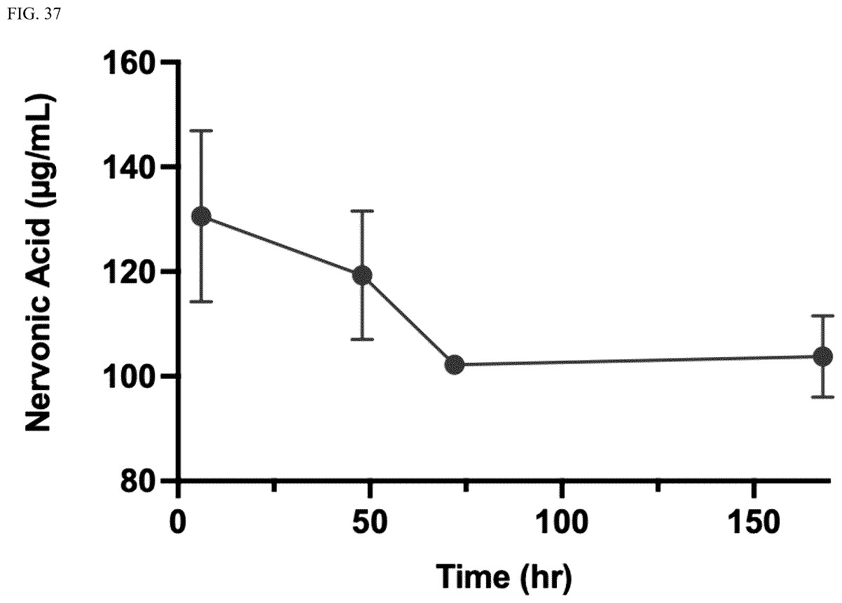

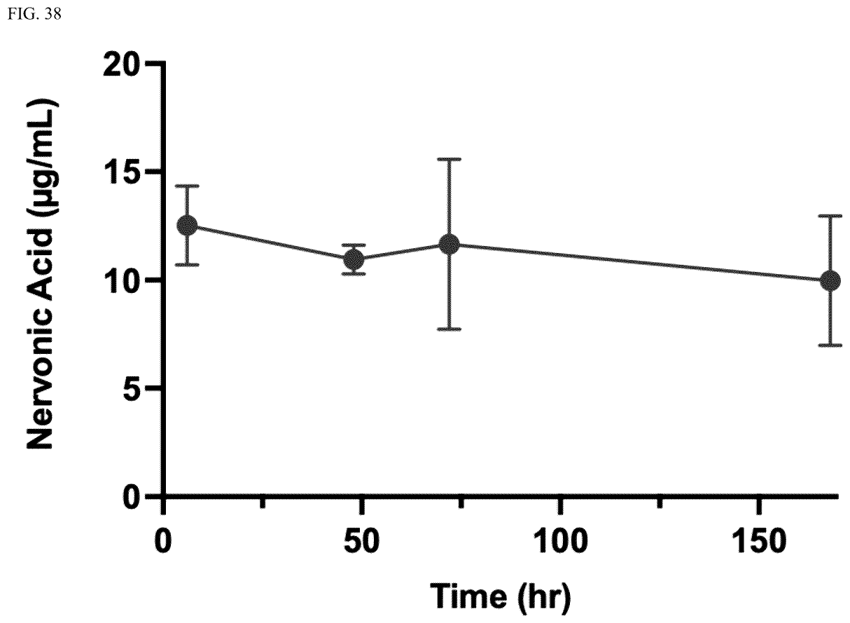

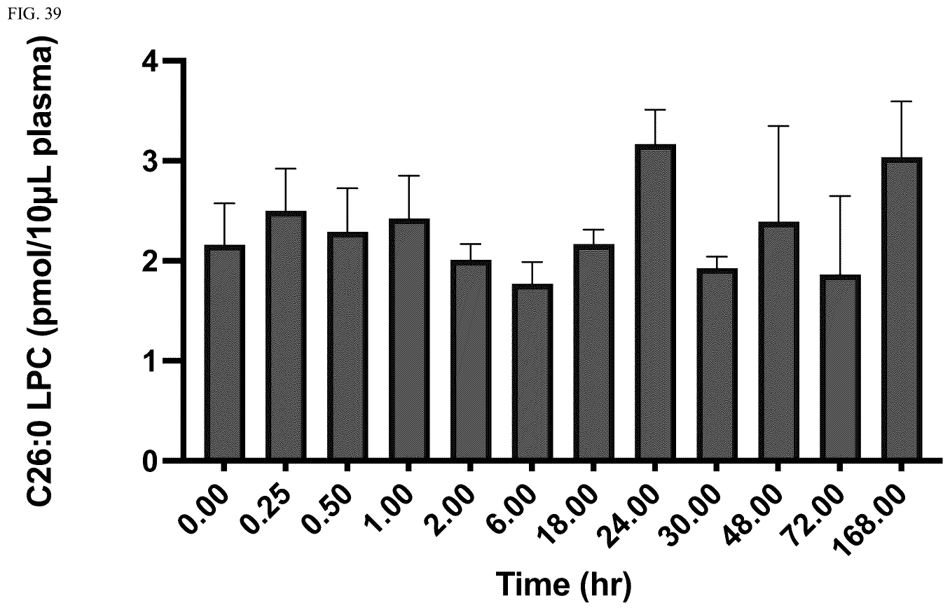

Pharmacokinetic studies were conducted to determine the key pharmacokinetic parameters of NA in an ALD mouse model and assess alterations in plasma C26:0 levels following single-dose NA administration. The pharmacokinetic parameters of NA were defined for the first time in rodents (FIG. 35-39). Single-dose NA administration does not significantly affect the levels of plasma C26:0 (FIG. 36) or plasma C26:0 LPC (FIG. 39). The concentrations of NA in the brain (FIG. 37) and in the liver (FIG. 38) were determined. Based on these results, the pharmacokinetic parameters were determined and summarized in Table 2.

TABLE 2
Non-compartmental analysis (NCA) estimated
pharmacokinetic parameters of NA.

| Parameter | Estimate (SE) | |
|---|---|---|
| Tₘₐₓ (hr) | 2 | |
| Cₘₐₓ (μg/mL) | 32.67 | (5.915) |
| AUC (hr*μg/mL) | 2332 | (213.1) |

| | |
|---|---|
| Ke (1/hr) | 0.01113 |
| CL/F (mL/hr) | 8.509 |
| V/F (mL) | 764.7 |
| t_{1/2} (hr) | 62.29 |

In the preceding description and following claims, the term “and/or” means one or all of the listed elements or a combination of any two or more of the listed elements; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

As used herein, the word “exemplary” means to serve as an illustrative example and should not be construed as preferred or advantageous over other embodiments. The words “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In the preceding description, particular embodiments may be described in isolation for clarity. Reference throughout this specification to “one embodiment,” “an embodiment,” “certain embodiments,” “one or more embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

For any method disclosed herein that includes discrete steps, the steps may be performed in any feasible order. And, as appropriate, any combination of two or more steps may be performed simultaneously.

As used herein, “have,” “has,” “having,” “include,” “includes,” “including,” “comprise,” “comprises,” “comprising” or the like are used in their open ended inclusive sense, and generally mean “include, but not limited to,” “includes, but not limited to,” or “including, but not limited to.” Further, wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, that which follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

### EXAMPLES

The present invention is illustrated by the following examples. It is to be understood that the particular examples, materials, amounts, and procedures are to be interpreted broadly in accordance with the scope and spirit of the invention as set forth herein.

### Nervonic Acid Formulations

An example of how some of the nervonic acid formulations of Table 1 were prepared is as follows. The desired mass of nervonic aid powder and cyclodextrin were mixed together via vertexing for five minutes. The desired amount of any second solvent (e.g., ethanol) was slowly added to the vortexed mixture and triturated in a round-bottom mortar and pestle to make a paste. The water was slowly added to the paste while stirring. The solution was transferred to a vessel amenable to shaking. The solution was incubated in an orbital shaker for two minutes at 37° C. Following shaking, the solution was sonicated four times at 30-second intervals. The sonicated solution was vortexed and incubated at 37° C. for two hours. The prepared formulation was stored at 4° C. until use.

### Imaging Study

Human dermal fibroblasts (GM17819) were seeded on chamber slides with a density of 12,500 cells per well and incubated at 37° C. At ˜80% confluence, cells were treated with ethanol (vehicle, <0.1%) and NA (5 μM, 20 μM, or 50 μM) for five days. Untreated AMN fibroblasts were considered the control group. After five days of incubation, cells were stained with various fluorogenic reagents for different purposes: MITOSOX Red (Life Technologies Corp., Carlsbad, CA; 5 μM) was used to visualize mitochondrial superoxide in live cells; CELLROX Green (Life Technologies Corp., Carlsbad, CA; 5 μM) was used to detect and quantify ROS in live cells; MITOTRACKER (Molecular Probes, Inc., Sunnyvale, CA) was used to detect mitochondrial activity; and 4′,6-diamidino-2-phenylindole (DAPI), a DNA binding dye (Sigma-Aldrich, St. Louis, MO), was also used. Cell localization and fluorescence visualization were then completed using a fluorescence microscope (BZ-X810; Keyence, Osaka, Japan). Phase-contrast images were also obtained.

### Seahorse Mitochondrial Function Study

Mitochondrial function was assessed on AMN cells (GM17819) using the Cell Mito Stress Test (CMST) in an XFe96 Extracellular Flux Analyzer (Agilent Technologies, Inc., Santa Clara, CA). Cells (1.25×10⁴ cells/well) were seeded overnight on Cell-Tak coated XFe96 microplate and then treated with NA (5 μM, 20 μM, or 50 μM) for 24 hours. For the CMST measurements, the culture medium was replaced with XF DMEM medium supplemented with glutamine 4 mM, sodium pyruvate 1 mM, and glucose 24.9 mM, pH 7.4 and 37° C. Oxygen consumption rate (OCR) was detected at baseline and after the following injections: 1) oligomycin (3 μM), 2) carbonyl cyanide 4-(trifluoromethoxy)phenylhydrazone/FCCP (2 μM), and 3) rotenone (1 μM) plus antimycin A (1 μM). This protocol allowed the analysis of the following parameters: basal respiration, ATP production, proton leak, maximal respiration, spare respiratory capacity, non-mito respiration, coupling efficiency percent, and spare respiratory capacity percent. OCR data were normalized by the number of cells determined by using CyQUANT NF Cell Proliferation Assay Kit (Thermo Fisher Scientific, Inc., Waltham, MA) and a fluorescence microplate reader. OCR results were generated by SEAHORSE WAVE PRO (Agilent Technologies, Inc., Santa Clara, CA) and analyzed with the SEAHORSE Analytics software (Agilent Technologies, Inc., Santa Clara, CA).

### In Vivo Mouse Study

FIG. 20 shows the experimental design for the in vivo mouse study. Double ALD knockout mice were grown to eight weeks. At eight week the mice were split into four treatment groups. Three of the treatment groups received nervonic acid (NA, 0.6% or 3%) or erucic acid (EA, 3%) through their daily diet. Specifically, mice were fed with rodent chow (TEKLAD Rodent Chow available from Inotiv in West Lafayette, IN) mixed with the desired amount of NA or EA. Food was available continuously and new food was added every other day. Daily food intake was weighed and mouse weighed every week. Blood samples were taken weekly for four weeks. Following four weeks, the mice were sacrificed, and their tissues analyzed for fatty acid content. GCMS was used for lipid fatty acid content analysis and LC-MS/MS was used to analyze Lyso-PC.

### Pharmacokinetics of NA Study

Pharmacokinetic studies were conducted using 12-week-old male ALD gene-knockout mice (KO; ABCD1-) following a single oral administration of 10 mg of NA. Blood samples were collected from the submandibular vein prior to dosing and at 0.25, 0.5, 1, 2, 6, 18, 24, 30, 48, 72, and 168 hours post-administration. The concentrations of NA in plasma and tissues were analyzed at the Kennedy Krieger Institute using GC/MS. Pharmacokinetic parameters including systemic clearance (CL/F), volumes of distribution (V/F), elimination half-life (ti/2), and the area under the curve (AUC) were estimated using PHOENIX (Certara USA, Radnor, PA) with non-compartmental analysis (NCA).

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A pharmaceutical composition comprising:
nervonic acid or a pharmaceutically acceptable salt thereof, and
a cyclodextrin.

2. The pharmaceutical composition of claim 1, wherein the cyclodextrin is a substituted or unsubstituted β-cyclodextrin, substituted or unsubstituted α-cyclodextrin; a substituted or unsubstituted γ-cyclodextrin; or any combination thereof.

3. The pharmaceutical composition of claim 1, wherein cyclodextrin comprises substituted or unsubstituted sulfobutylether-β-cyclodextrin.

4. The pharmaceutical composition of claim 3, wherein the average degree of substitution is 6.0 to 8.0.

5. The pharmaceutical composition of claim 1, wherein the pharmaceutical composition is a drug product.

6. The pharmaceutical composition of claim 1, wherein the pharmaceutical composition is in a solid dosage form.

7. The pharmaceutical composition of claim 1, wherein the pharmaceutical composition is in a liquid dosage form.

8. A method of treating a subject having or at risk of having adrenoleukodystrophy, a neurological disorder, or both, the method comprising administering a pharmaceutical composition to the subject, the pharmaceutical composition comprising:
nervonic acid or a pharmaceutically acceptable salt thereof, and
a cyclodextrin.

9. The method of claim 8, wherein the pharmaceutical composition is administered prior to the subject showing symptoms of adrenoleukodystrophy.

10. The method of claim 8, wherein adrenoleukodystrophy is of the childhood cerebral adrenoleukodystrophy phenotype, adrenomyeloneuropathy phenotype, Addison disease phenotype, or any combination thereof.

11. The method of claim 9, wherein adrenoleukodystrophy is of the childhood cerebral adrenoleukodystrophy phenotype, adrenomyeloneuropathy phenotype, Addison disease phenotype, or any combination thereof.

12. The method of claim 8, wherein the pharmaceutical composition is administered in multiple doses.

13. A method of reducing accumulation of one or more very long chain fatty acid in a cell, the method comprising contacting the cell with a composition comprising:
nervonic acid or a pharmaceutically acceptable salt thereof, and
a cyclodextrin.

14. The method of claim 13, wherein the one or more very long chain fatty acid is hexacosanoic acid (C26:0) and/or C26:0-lysophsphatidylcholin.

15. The method of claim 13, wherein the pharmaceutical composition is administered in multiple doses.

16. A method of improving mitochondrial function in a cell, the method comprising contacting the cell with a composition comprising:
nervonic acid or a pharmaceutically acceptable salt thereof, and
a cyclodextrin.

17. The method of claim 16, wherein improvement of mitochondrial function is indicated by an increase in ATP production, an increase in basal respiration, and increase in maximal respiration, an increase in spare respiratory capacity, a decrease in reactive oxygen species, and/or a decrease in mitochondrial superoxide.

18. The method of claim 16, wherein the pharmaceutical composition is administered in multiple doses.
